SEQUENCE LISTING (16 sequences)

SEQ ID NO: 1 - DNA, 23 bp, Artificial
  Human/rat CCK1 upstream cDNA primer
ctgctcaaggatttcatcttcgg

SEQ ID NO: 2 - DNA, 20 bp, Artificial
  Human/rat downstream cDNA primer
gggaaggtggccatgaagcc

SEQ ID NO: 3 - DNA, 26 bp, Artificial
  Canine CCK1 upstream RT-PCR primer
catttccttcatcctcctgctgtcct

SEQ ID NO: 4 - DNA, 21 bp, Artificial
  Canine CCK1 downstream RT-PCR primer
cgctcaggggcccggggccga

SEQ ID NO: 5 - DNA, 39 bp, Artificial
  Canine CCK1 5′ end cDNA primer
aacgttggaattcgccaccatggaggtggccgacagcct

SEQ ID NO: 6 - DNA, 37 bp, Artificial
  Canine CCK1 3′ end cDNA primer
aacgttgcggccgctcaggggcccggggccgaggcgc

SEQ ID NO: 7 - DNA, 20 bp, Artificial
  Canine CCK1 RT-PCR forward primer
catctacagcaacctggtgc

SEQ ID NO: 8 - DNA, 20 bp, Artificial
  Canine CCK1 RT-PCR reverse primer
gtggacagctgccggagctc

SEQ ID NO: 9 - DNA, 22 bp, Artificial
  B-actin 5′ upstream primer
catgggccagaaggactcctac

SEQ ID NO: 10 - DNA, 20 bp, Artificial
  B-actin 3′ downstream primer
cacgctccgtgaggatcttc

SEQ ID NO: 11 - DNA, 1287 bp, Canine
atggaggtggccgacagcctgcttgggaatggcagcgacgtccccccgccctgtgagctggggctcgagaacgagaccctggtctgcctggagcagccccgcgccgccaaagagtggcagccggctgtgcagatcctcctgtattccctgattttcctgctcagcgtgctggggaacacgctggtcatcacggtgctcattcggaacaagaggatgcgcaccgtcaccaacatcttcctgctgtccctggccgtcagcgacctcatgctgtgcctcttctgcatgcccttcaacctcatccccaacctgctcaaggatttcatcttcgggagcgccgtctgcaagaccaccacctacttcatgggcacgtcggtgagcgtatccacctttaacctggtagccatatctctggaaagatacggtgcgatttgcaaacccttacagtcccgggtctggcagacgaaatcccacgctttgaaggtgatcgccaccacctggtgcctgtcctttaccatcatgactccctaccccatctacagcaacctggtgccttttaccaaaactaacaaccagacggcgaacatgtgccgctttctactgccaaatgatgtgatgcagcagtcctggcacacgttcctgttactcatcctctttcttattcctggaattgtgatgatggtggcatacggattgatctctttggaactttaccaaggaataaaatttgatgctatccagaagaagtctgctagggacaggaacccgagcaccggcagcagcggcaggtatgaggacggcgacggctgttacctgcagaaggccaggccccgccggaggctggagctccggcagctgtccacccccggcagcggcaggctcaaccgcatcaggagcaccagctctacggccaacctgatggccaagaagcgggtgatccgcatgctcatggtcatcgtggtcctcttcttcctgtgctggatgcccatcttcagcgccaacgcctggcgggcctacgacacggcctctgcygagcgccgcctctcggggacccccatttccttcatcctcctgctgtcctatacctcctcctgcgtcaaccccatcatctactgcttcatgaacaagcgcttccgcctcggcttcctggccaccttcccctgctgcccccaccccggtcccccagggccgagaggcgaggtgggagaggaggaggaaggcaggaccacgggggcctctctgtccaggtactcctacagccacatgagcgcctcggccccgggcccctga

SEQ ID NO: 12 - DNA, 1287 bp, Canine
atggaggtggccgacagcctgcttgggaatggcagcgacgtccccccgccctgtgagctggggctcgagaacgagaccctggtctgcctggagcagccccgcgccgccaaagagtggcagccggctgtgcagatcctcctgtattccctgattttcctgctcagcgtgctggggaacacgctggtcatcacggtgctcattcggaacaagaggatgcgcaccgtcaccaacatcttcctgctgtccctggccgtcagcgacctcatgctgtgcctcttctgcatgcccttcaacctcatccccaacctgctcaaggatttcatcttcgggagcgccgtctgcaagaccaccacctacttcatgggcacgtcggtgagcgtatccacctttaacctggtagccatatctctggaaagatacggtgcgatttgcaaacccttacagtcccgggtctggcagacgaaatcccacgctttgaaggtgatcgccaccacctggtgcctgtcctttaccatcatgactccctaccccatctacagcaacctggtgccttttaccaaaactaacaaccagacggcgaacatgtgccgctttctactgccaaatgatgtgatgcagcagtcctggcacacgttcctgttactcatcctctttcttattcctggaattgtgatgatggtggcatacggattgatctctttggaactttaccaaggaataaaatttgatgctatccagaagaagtctgctagggacaggaacccgagcaccagcagcagcggcaggtatgaggacggcgacggctgttacctgcagaaggccaggccccgccggaggctggagctccggcagctgtccacccccggcagcggcaggctcaaccgcatcaggagcaccagctctacggccaacctgatggccaagaagcgggtgatccgcatgctcatggtcatcgtggtcctcttcttcctgtgctggatgcccatcttcagcgccaacgcctggcgggcctacgacacggcctctgcygagcgccgcctctcggggacccccatttccttcatcctcctgctgtcctatacctcctcctgcgtcaaccccatcatctactgcttcatgaacaagcgcttccgcctcggcttcctggccaccttcccctgctgcccccaccccggtcccccagggccgagaggcgaggcgggagaggaggaggaaggcaggaccacgggggcctctgtgtccaggtactcctacagccacatgagcgcctcggccccgggcccctga

SEQ ID NO: 13 - DNA, 1287 bp, Canine
atggaggtggccgacagcctgcttgggaatggcggcgacgtccccccgccctgtgagctggggctcgagaacgagaccccggtctgcctggagcagccccgcgccgccaaagagtggcagccggctgtgcagatcctcctgtattccctgattttcctgctcagcgtgctggggaacacgctggtcatcacggtgctcattcggaacaagaggatgcgcaccgtcaccaacatcttcctgctgtccctggccgtcagcgacctcatgctgtgcctcttctgcatgcccttcaaccccatccccaacctgctcaaggatttcatcttcgggagcgccgtctgcaagaccaccacctacttcatgggcacgtcggtgagcgtatccacctttaacctggtagccatatctctggaaagatacggtgcgatttgcaaacccttacagtcccgggtctggcagacgaaatcccacgctttgaaggtgatcgccaccacctggtgcctgtcctttaccatcatgactccctaccccatctacagcaacctggtgccttttaccaaaactaacaaccagacggcgaacatgtgccgctttctactgccaaatgatgtgatgcagcagtcctggcacacgttcctgttactcatcctctttcttattcctggaattgtgatgatggtggcatacggattgatctctttggaactttaccaaggaataaaatttgatgctatccagaagaagtctgctagggacaggaacccgagcaccagcagcagcggcaggtatgaggacggcgacggctgttacctgcagaaggccaggccccgccggaggctggagctccggcagctgtccacccccggcagcggcaggctcaaccgcatcaggagcaccagctctacggccaacctgatggccaagaagcgggtgatccgcatgctcatggtcatcgtggtcctcttcttcctgtgctggatgcccatcttcagcgccaacgcctggcgggcctacgacacggcctctgcygagcgccgcctctcggggacccccatttccttcatcctcctgctgtcctatacctcctcctgcgtcaaccccatcatctactgcttcatgaacaagcgcttccgcctcggcttcctggccaccttcccctgctgcccccaccccggtcccccagggccgagaggcgaggcgggagaggaggaggaaggcaggaccacgggggcctctgtgtccaggtactcctacagccacatgagcgcctcggccccgggcccctga

SEQ ID NO: 14 - protein, 428 aa, Canine
MEVADSLLGNGSDVPPPCELGLENETLVCLEQPRAAKEWQPAVQILLYSLIFLLSVLGNTLVITVLIRNKRMRTVTNIFLLSLAVSDLMLCLFCMPFNLIPNLLKDFIFGSAVCKTTTYFMGTSVSVSTFNLVAISLERYGAICKPLQSRVWQTKSHALKVIATTWCLSFTIMTPYPIYSNLVPFTKTNNQTANMCRFLLPNDVMQQSWHTFLLLILFLIPGIVMMVAYGLISLELYQGIKFDAIQKKSARDRNPSTGSSGRYEDGDGCYLQKARPRRRLELRQLSTPGSGRLNRIRSTSSTANLMAKKRVIRMLMVIVVLFFLCWMPIFSANAWRAYDTASAERRLSGTPISFILLLSYTSSCVNPIIYCFMNKRFRLGFLATFPCCPHPGPPGPRGEVGEEEEGRTTGASLSRYSYSHMSASAPGP

SEQ ID NO: 15 - protein, 428 aa, Canine
MEVADSLLGNGSDVPPPCELGLENETLVCLEQPRAAKEWQPAVQILLYSLIFLLSVLGNTLVITVLIRNKRMRTVTNIFLLSLAVSDLMLCLFCMPFNLIPNLLKDFIFGSAVCKTTTYFMGTSVSVSTFNLVAISLERYGAICKPLQSRVWQTKSHALKVIATTWCLSFTIMTPYPIYSNLVPFTKTNNQTANMCRFLLPNDVMQQSWHTFLLLILFLIPGIVMMVAYGLISLELYQGIKFDAIQKKSARDRNPSTSSSGRYEDGDGCYLQKARPRRRLELRQLSTPGSGRLNRIRSTSSTANLMAKKRVIRMLMVIVVLFFLCWMPIFSANAWRAYDTASAERRLSGTPISFILLLSYTSSCVNPIIYCFMNKRFRLGFLATFPCCPHPGPPGPRGEAGEEEEGRTTGASVSRYSYSHMSASAPGP

SEQ ID NO: 16 - protein, 428 aa, Canine
MEVADSLLGNGGDVPPPCELGLENETPVCLEQPRAAKEWQPAVQILLYSLIFLLSVLGNTLVITVLIRNKRMRTVTNIFLLSLAVSDLMLCLFCMPFNPIPNLLKDFIFGSAVCKTTTYFMGTSVSVSTFNLVAISLERYGAICKPLQSRVWQTKSHALKVIATTWCLSFTIMTPYPIYSNLVPFTKTNNQTANMCRFLLPNDVMQQSWHTFLLLILFLIPGIVMMVAYGLISLELYQGIKFDAIQKKSARDRNPSTSSSGRYEDGDGCYLQKARPRRRLELRQLSTPGSGRLNRIRSTSSTANLMAKKRVIRMLMVIVVLFFLCWMPIFSANAWRAYDTASAERRLSGTPISFILLLSYTSSCVNPIIYCFMNKRFRLGFLATFPCCPHPGPPGPRGEAGEEEEGRTTGASVSRYSYSHMSASAPGP

(19) United States
(12) Patent Application Publication
(10) Pub. No.: US 2008/0124741 A1
(43) Pub. Date: May 29, 2008

(54) Canine Cholecystokinin 1 Receptor Materials And Their Use

(76) Inventors: Heng Dai, San Diego, CA (US); Magda F. Morton, San Diego, CA (US); Jayashree Pyati, San Diego, CA (US); Nigel P. Shankley, Solan Beach, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/664,383

(22) PCT Filed: Oct. 11, 2005
(86) PCT No.: PCT/US2005/036476

§ 371 Date: Mar. 29, 2007

Related U.S. Application Data
(60) Provisional application No. 60/617,888, filed on Oct. 12, 2004.

(51) Int. Cl. G01N 33/567; C07K 14/00; C12N 15/11; C12N 15/00; C12N 5/06

(52) U.S. Cl. 435/7.21; 530/399; 536/23.51; 435/320.1; 435/325

(57) ABSTRACT

Canine CCK1 receptor materials are described, such as polypeptides having amino acid sequences corresponding to SEQ ID Nos.: 14, 15, and 16 or functional variants thereof and polynucleotides expressing them having nucleic acid sequences corresponding to SEQ ID Nos.: 11, 12, and 13 or complements thereof. Such materials are useful as reagents in drug screening assays to identify compounds having CCK1R-modulating activity.

This application claims priority to U.S. Provisional Application No. 60/617,888, filed Oct. 12, 2004.

### FIELD OF THE INVENTION

The present invention generally relates to canine cholecystokinin 1 (CCK1 or CCK_{A}) receptor materials, including polypeptides and polynucleotides encoding polypeptides, and associated vectors and recombinant host cells. The invention also relates to methods of using such materials to assay compounds for their CCK1 modulating activity.

### BACKGROUND OF THE INVENTION

Cholecystokinin (CCK) receptors, which are G protein-coupled receptors, are widely distributed throughout the gastrointestinal and central nervous systems, where they regulate pancreatic and gastric secretion, smooth muscle motility, growth, anxiety, satiety, pain or analgesia, and neuroleptic activity. See U.S. Pat. No. 6,169,173. CCK receptors were originally classified into two sub-types, CCK1 (formerly CCK_{A}) and CCK2 (formerly CCK_{B} or gastrin receptor), on the basis of differences in agonist rank potency orders and through the use of receptor-selective antagonists (see, e.g., Noble et al., 1999, Pharmacol. Rev., 51:745-781). Subsequently, both of these receptors were cloned from a number of species and it was shown that there was a high degree of sequence homology across species (84-93% for the CCK2 receptor and 87-92% for the CCK1 receptor in humans, guinea pig, rat and rabbit).

Notwithstanding this conservation of amino-acid sequence, species variation in the pharmacological profiles of some CCK receptor ligands has been demonstrated. For example, a single amino-acid substitution in the CCK2 receptor has been shown to account for the reverse selectivity of the non-peptide antagonists L-365,260 and L-364,718 between dog and human CCK receptors (Beinborn et al., 1993, Nature, 362:348-350). Similarly, differences have been demonstrated in the expression of efficacy by the partial agonists PD135,158 and L-740,093 between the mouse, human and dog receptor, which were subsequently attributed to specific amino-acid substitutions (Kopin et al., 1997, Proc. Natl. Acad. Sci. U.S.A., 94:11043-11048). Thus, synthetic ligands can differentiate between species variants of the same receptor protein.

The actions of CCK and gastrin in the canine gastrointestinal tract have been investigated extensively due to the physiological and structural similarity of the canine and human gut. The non-peptide antagonist, L-364,718, is a high affinity and selective human CCK1 receptor antagonist which has been used a pharmacological tool to delineate the contributions of the CCK/gastrin receptor family to many physiological functions, including transient lower esophageal sphincter relaxation (Boulant et al., 1994, Gastroenterology, 107:1059-1066), intestinal transit time (Lin et al., 2002, Dig. Dis. Sci., 47:2217-2221), pancreatic growth and secretion (U.S. Pat. No. 6,169,173; Niebergall-Roth et al., 1997, Am. J. Physiol., 272:G1550-G1559), gallbladder contraction (Sonobe et al., 1995, Regul. Pept., 60:33-46) and gastric antral motility and gastric emptying (Tanaka et al., 1999, Dig. Dis. Sci., 44:1516-1524). However, the interpretation of these data has been limited by the lack of canine CCK1 receptor materials and therefore the absence of affinity values for this compound at canine receptors. There is therefore a need to identify such canine CCK1 receptors.

### SUMMARY OF THE INVENTION

In one general aspect, the invention is directed to an isolated biologically active canine cholecystokinin 1 receptor polypeptide having an amino acid sequence as set forth in SEQ ID NO.:14 or SEQ ID NO.:15 or a functional variant thereof. Preferably, the polypeptide has an amino acid sequence as set forth in SEQ ID NO.:14 or SEQ ID NO.:15. The invention is also generally directed to a CCK1 polypeptide having an amino acid sequence as set forth in SEQ ID NO.:16.

Another general aspect of the invention relates to isolated polynucleotides encoding the above-described CCK1 receptor polypeptides. Thus, the invention is directed to a polynucleotide encoding a canine cholecystokinin 1 receptor polypeptide, where the polynucleotide has a sequence as set forth in SEQ ID NO.:11 or SEQ ID NO.:12 or is a complement thereof that hybridizes under stringent conditions thereto. Preferably, the polynucleotide has a nucleic acid sequence as set forth in SEQ ID NO.:11 or SEQ ID NO.:12. Additionally, the invention generally relates to an isolated polynucleotide encoding a canine cholecystokinin 1 receptor polypeptide, where the polynucleotide has the nucleotide sequence set forth in SEQ ID NO.:13 or is a complement thereof that hybridizes under stringent conditions thereto.

In other general aspects, the invention is directed to vectors each comprising one of the polynucleotides as described above operably linked to a promoter element that produces the canine cholecystokinin 1 receptor RNA or expresses the canine cholecystokinin 1 receptor polypeptide encoded by the polynucleotide in a transfected host cell.

In additional general aspects, the invention is directed to recombinant host cells transfected with one of the vectors as described above.

In further general aspects, the invention pertains to methods for identifying a compound that modulates a biological activity of a biologically active canine cholecystokinin 1 receptor or a functional variant thereof. One such method comprises: (a) contacting a test sample comprising a compound with an assay reagent comprising the receptor and a cholecystokinin 1 receptor ligand; (b) determining the biological activity of the receptor after performing step (a); and (c) comparing the biological activity determined in step (b) with a control measurement obtained by contacting a control sample not containing the compound with the assay reagent. Another such method comprises: (a) contacting a biologically active canine CCK1 receptor with a test compound and with a labeled ligand for the receptor; (b) determining the amount of the labeled ligand that complexes with the receptor; and (c) comparing the amount determined in step (b) with a control measurement obtained by contacting the receptor with the labeled ligand in the absence of the test compound. An additional method is a whole cell assay for detecting modulation of the canine CCK1 receptor by steps comprising: (a) contacting the compound and a cell that contains biologically active CCK1 receptor or a variant thereof; and (b) measuring for change in the cell in response to modified receptor function by the compound. In preferred embodiments of such methods, the CCK1 receptor material used in the assay is a component of a biological sample derived from a dog.

Other aspects and features of the invention will be apparent from the detailed description below with reference to the drawing figures.

### BRIEF DESCRIPTION OF THE DRAWINGS

FIG. 1 illustrates the location of primers and estimated size of PCR products used in the amplification of the canine CCK1 receptor. UP1, UP2 and UP3 are upstream or sense primers and DN1, DN2 and DN3 are the downstream or antisense primers. The sequences of the primers are listed in Table 1.

FIG. 2 illustrates the PCR products amplified from canine gallbladder cDNA. Lane 1, size markers generated using a combination of lamda-3 fragments; lane 2, 845-bp PCR product of primers UP1 and DN1; lane 2, 227-bp, 3′ end of sequence amplified using primers UP2 and DN2; lane 3, full-length cDNA of canine CCK1 receptor (1287 bp) amplified using primers UP3 and DN3.

FIG. 3 depicts the nucleotide and amino acid sequences of the canine CCK1 receptor. The putative membrane spanning segments are underlined and marked TM (transmembrane) I-VII. The nucleotide and amino acid polymorphisms that were identified during the cloning are marked 1-6, with specific base pairs shaded grey. Alterations 4-6 were found in variant #1 (SEQ ID NO.:12 and 15; polynucleotide and amino acid sequences, respectively) and all six polymorphisms were found in variant #2 SEQ ID NO.:13 and 16; polynucleotide and amino acid sequences, respectively).

FIG. 4 provides a comparison of the amino-acid (a.a.) sequences of the canine, human (Genbank accession number 113605) and rat CCK1 receptors (Genbank accession number M88096). Putative membrane spanning regions are underlined.

FIG. 5 depicts the RT-PCR products of full-length canine CCK1 receptor (primer UP3 and DN3) amplified from different canine tissues (from left to right: gastric antrum, gallbladder, colon, kidney, liver, spleen, hypothalamus and thalamus). To confirm integrity of cDNA, βactin primers were also used on each sample to amplify this housekeeping gene.

FIGS. 6A-6E illustrate competition between [¹²⁵I]-BH-CCK-8S (20 pM) and increasing concentrations of L-364,718 (FIG. 6A), L-365,260 (FIG. 6B), dexloxiglumide (FIG. 6C), YF476 (FIG. 6D), and YM022 (FIG. 6E) at the canine CCK1, human CCK1 and canine CCK2 receptors. Total binding and non-specific binding were defined with 50 μl assay buffer and 50 μl of 10 μM 2-NAP, respectively. Data represent the mean ±s.d. (standard deviation) mean of three experiments.

FIGS. 7A-7C show total, non-specific, and specific binding of [¹²⁵I]-BH-CCK-8S (20 pM) plotted as a function of increasing protein concentration at the wild-type (FIG. 7A), variant #1 (FIG. 7B), and variant #2 (FIG. 7C) canine CCK1 receptors. Wild type and variant receptors were transiently transfected into HEK cells and the protein concentration determined after membrane preparation (BCA kit, Pierce).

FIGS. 8A and 8B illustrate results of a saturation analysis of the binding of [¹²⁵I]-BH-CCK-8S to the wild-type CCK1 receptor. Increasing concentrations of [¹²⁵I]-BH-CCK-8S were incubated with 80 μg ml⁻¹ of protein. FIG. 8A illustrates the biphasic nature of the data. FIG. 8B illustrates the first phase of the saturation used for analysis (shown in grey box in FIG. 8A). Data are representative of three experiments.

### DETAILED DESCRIPTION OF INVENTION AND ITS PREFERRED EMBODIMENTS

For the sake of brevity, the disclosures of all publications cited herein are incorporated by reference. Unless defined otherwise herein or as apparent from the context, all technical and scientific terms used herein have the same meaning as used in the art.

The following are abbreviations that are at times used in this specification: bp=base pair; BH=Bolton-Hunter conjugated; CCK=cholecystokinin; CCKR═CCK receptor; cpm=counts per minute; cAMP=cyclic adenosine monophosphate; cDNA=complementary DNA; kb=kilobase (1000 base pairs); kDa=kilodalton; G protein=GTP-binding protein; GTP=guanosine 5′-triphosphate; nt=nucleotide; PAGE=polyacrylamide gel electrophoresis; PCR=polymerase chain reaction.

The terms “including,” “comprising” and “containing” are used herein in their open, non-limiting sense.

The canine CCK1 receptor has now been cloned, and its expression and pharmacological characterization investigated. As described in the examples below, the cholecystokinin-1 receptor was amplified from canine gallbladder tissue using human CCK1 receptor specific primers. The sequence of the fragment was used in conjunction with the canine genomic sequence to design canine specific primers for the cloning of the canine CCK1 receptor. The cloned wild-type receptor, found to be 89% identical to the human and 85% identical to the rat CCK1 receptor, was expressed in CHO-K cells for pharmacological characterization. Five structurally-diverse, CCK-receptor selective, ligands were used in radioligand binding studies with [¹²⁵I]-BH-CCK-8S as radioligand. The affinity values estimated for these ligands, L-364,718, L-365,260, YF476, YM022 and dexloxiglumide, were not significantly different between the human and canine CCK1 receptors. In addition, the selectivity of these compounds between canine CCK1 and canine CCK2 receptors was consistent with the selectivity between the human forms of these receptors. During the cloning of the canine CCK1 receptor, two additional variant forms of the receptor were identified. These variants had three (variant #1) and six (variant #2) amino-acid differences compared to the wild-type canine CCK1 receptor. Only variant #1 was found to bind [¹²⁵I]-BH-CCK-8S and this form of the receptor displayed an identical pharmacological profile to the wild-type receptor.

Accordingly, certain general aspects of the invention relate to isolated biologically active cholecystokinin 1 receptor polypeptides and functional variants thereof, polynucleotides that encode them, expression vectors comprising such polynucleotides, and recombinant host cells transfected or transformed by such vectors.

“Polypeptide” refers to a peptidic molecule comprising two or more amino acids joined to each other in a linear chain by peptide bonds. As used herein, the term refers both to short chains, which are also referred to in the art as, e.g., peptides, oligopeptides and oligomers, and to longer chains, which are often referred to in the art as proteins, of which there are many types.

A “biologically active” polypeptide or polynucleotide refers to a molecule that is active as determined in vivo or in vitro according to standard or conventional or accepted techniques. Such activities can be a direct activity, such as an association with or an enzymatic activity on a second protein, or an indirect activity, such as a cellular signaling activity mediated by interaction of the protein with a second protein. For example, an illustrative biological activity of a CCK1 receptor ligand, such as CCK-8, is its ability to bind or form a complex with a CCK1 receptor and initiate one or more signal transduction events conducted thereby. An exemplary biological activity of canine CCK1 receptor is that, upon binding to a ligand for the receptor, it activates a chain of events that alters the concentration of intracellular signaling molecules (second messenger molecules), such as cyclic AMP and calcium via activating G-protein, which has a high affinity to GTP. These intracellular signaling molecules in turn alter the physiology and behavior of the cell.

With respect to the canine CCK1R polypeptides described herein, functional variants may be determined by making one or more modifications to a polypeptide and testing the biological activity of the resulting variant. For example, as understood in the art, polypeptides often contain amino acids other than the twenty amino acids commonly referred to as the naturally occurring amino acids, and many amino acids, including the terminal amino acids, can be modified in a given polypeptide, either by natural processes, such as processing and other post-translational modifications, and by known chemical modification techniques. Common modifications that occur naturally in polypeptides are too numerous to list exhaustively here, but are described in basic texts and in more detailed monographs, as well as in research literature, and are therefore within the purview of persons of ordinary skill in the art. Among the known modifications which can be present in polypeptides of the present invention include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Several common modifications, such as glycosylation, lipid attachment, sulfation, gamma-carboxylation of glutamic acid residues, hydroxylation and ADP-ribosylation, are described in many basic texts, including PROTEINS—STRUCTURE AND MOLECULAR PROPERTIES, 2nd ed., T. E. Creighton, W.H. Freeman and Company, New York (1993). Many reviews are also available on this subject, such as those provided by Wold, “Posttranslational Protein Modifications: Perspectives and Prospects,” pgs. 1-12 in POSTTRANSLATIONAL COVALENT MODIFICATION OF PROTEINS, Johnson (ed.), Academic Press, New York (1983); Seifter et al., 1990, Meth. Enzymol., 182:626-646; and Rattan et al., 1992, “Protein Synthesis: Posttranslational Modifications and Aging”, Ann. N.Y. Acad. Sci. 663:48-62.

It will be appreciated, as is known and as noted above, that polypeptides are not always entirely linear. For instance, polypeptides can be post-translationally modified, including via natural processing or through human manipulation. Circular, branched and branched-circular polypeptides can be synthesized by non-translation natural processes and by entirely synthetic methods as well. Modifications can occur anywhere in a polypeptide, including the peptide backbone, the amino acid side-chains, and the amino or carboxyl termini. For example, blockage of the amino or carboxyl group or both in a polypeptide by a covalent modification is common in naturally occurring and synthetic polypeptides, and such modifications can be present in polypeptides of the present invention. For instance, the amino terminal residue of polypeptides made in E. coli or other cells, prior to proteolytic processing, will typically be N-formylmethionine. During post-translational modification of the peptide, a methionine residue at the NH₂-terminus can be deleted. Accordingly, the methionine-containing and the methionineless amino terminal variants of a protein may be prepared.

The modifications that occur in a polypeptide often will be a function of how it is made. For polypeptides made by expressing a cloned gene in a host, for instance, the nature and extent of the modifications may be determined by the host cell posttranslational modification capacity and the modification signals present in the polypeptide amino acid sequence. For instance, as is known, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect-cell expression systems have been developed to express efficiently mammalian proteins having native patterns of glycosylation, among other things. Similar considerations apply to other modifications. It will be appreciated that the same type of modification can be present in the same or varying degree at several sites in a given polypeptide. Also, a given polypeptide can contain many types of modifications. Thus, variants encompass all such modifications, including those that are present in polypeptides synthesized recombinantly by expressing a polynucleotide in a host cell.

An “isolated” polypeptide is a polypeptide substantially free of or separated from cellular material or other contaminating proteins from the cell or tissue source from which the polypeptide is produced and isolated, or substantially free of chemical precursors or other chemicals when the polypeptide is chemically synthesized. For example, protein that is substantially free of cellular material can include preparations of protein having less than about 30%, or preferably 20%, or more preferably 10%, or even more preferably 5%, or yet more preferably 1% (by dry weight), of contaminating proteins.

In preferred embodiments, the isolated polypeptide is substantially pure. Thus, when the protein or biologically active portion thereof is recombinantly produced, it is substantially free of culture medium, e.g., culture medium representing less than about 20%, or more preferably 10%, or even more preferably 5%, or yet more preferably 1%, of the volume of the protein preparation. When the protein is produced by chemical synthesis, it is substantially free of chemical precursors or other chemicals, i.e., it is separated from chemical precursors or other chemicals that are involved in the synthesis of the protein. Accordingly such preparations of the polypeptide have less than about 30%, or preferably 20%, or more preferably 10%, or even more preferably 5%, or yet more preferably 1% (by dry weight), of chemical precursors or compounds other than the polypeptide of interest.

Isolated polypeptides can have several different physical forms. The isolated polypeptide can exist as a full-length nascent or unprocessed polypeptide, or as partially processed polypeptides or combinations of processed polypeptides. The full-length nascent polypeptide can be post-translationally modified by specific proteolytic cleavage events that result in the formation of fragments of the full-length nascent polypeptide. A fragment, or physical association of fragments, can have the biological activity associated with the full-length polypeptide; of course, the degree of biological activity associated with individual fragments can vary.

Polypeptides of the invention may be prepared using polynucleotides of the invention. The term “polynucleotide” as used herein refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single- or double-stranded. However, linkages may include any of the linkages known in the art, including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

An “isolated” polynucleotide is one that is substantially separated from or free of nucleic acid molecules with differing nucleic acid sequences. Embodiments of the isolated polynucleotide molecule of the invention include cDNA and genomic DNA and RNA, antisense RNA. Preferred polynucleotides are obtained from biological samples derived from a dog, such as from blood samples or tissue specimens.

A “functional variant” refers to a modified form, homolog, or variant of a designated polypeptide or a polynucleotide encoding such polypeptide that possesses essentially the same biological activity as the designated one. Functional variants may be the product of, e.g., a polymorphism, a truncation, or a fragmentation, of the polypeptide or polynucleotide. For example, the sequence corresponding to SEQ ID NO:15 is a variant of the cholecystokinin 1 receptor corresponding to SEQ ID NO.14. “Polymorphism” refers to a set of genetic variants at a particular genetic locus among individuals in a population.

Variants of a polynucleotide may be their complements. For example, a complement that hybridizes under stringent conditions to a particular polynucleotide may be a useful functional variant of it. An extensive guide to the hybridization of nucleic acids is found in Sambrook et al., MOLECULAR CLONING: A LABORATORY MANUAL, Second Edition, Cold Spring Harbor Laboratory, Cold Spring Harbor, N.Y. (1989), and Tijssen, TECHNIQUES IN BIOCHEMISTRY AND MOLECULAR BIOLOGY—HYBRIDIZATION WITH NUCLEIC PROBES, “Overview of principles of hybridization and the strategy of nucleic acid assays” (1993). Stringent hybridization conditions may be suitably selected in view of the particular sequence. Exemplary stringent conditions include a temperature of about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Exemplary stringent conditions further include a salt concentration less than about 1.0 M sodium ion, e.g., about 0.01 to 1.0 M sodium ion concentration (or other salts) at pH 7.0 to 8.3 and a temperature of at least about 30° C. for short probes (e.g., 10 to 50 nucleotides) and at least about 60° C. for long probes (e.g., greater than 50 nucleotides). Stringent conditions may also include the addition of destabilizing agents such as formamide. For selective or specific hybridization, an exemplary positive signal is at least two times background, optionally 10 times background hybridization. Illustrative stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C. Such washes can be performed for 5, 15, 30, 60, 120, or more minutes.

Canine CCK1 receptor polynucleotides may be inserted into expression vectors for introduction of such polynucleotides into host cells for the expression, i.e., production of the encoded mRNA or protein, of the canine CCK1 receptor polypeptides encoded by such polynucleotides in such host cells. The expressed canine CCK1 receptor polypeptides from the resulting recombinant host cells are isolated for various uses in vitro, or serve to modulate various other in vivo activities within such recombinant host cells.

The term “vector” refers to a nucleic acid molecule capable of transporting another nucleic acid to which it has been linked. One type of vector is a plasmid, which refers to a circular double-stranded DNA loop into which additional DNA segments can be inserted. Another type of vector is a viral vector wherein additional DNA segments can be inserted. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors—expression vectors—are capable of directing the expression of genes to which they are operably linked. Vectors of utility in recombinant DNA techniques may be in the form of plasmids. Alternatively, other forms of vectors, such as viral vectors (e.g., replication defective retroviruses, adenoviruses and adeno-associated viruses), which serve equivalent functions, may be used.

A “host cell” refers to a cell that contains a DNA molecule either on a vector or integrated into a cell chromosome. A host cell can be either a native host cell that contains the DNA molecule endogenously or a recombinant host cell.

One example of a host cell is a recombinant host cell, which is a cell that has been transformed or transfected by an exogenous DNA sequence. A cell has been transformed by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA. A “clone” is a population of cells derived from a single cell or common ancestor by mitosis. A “cell line” is a clone of a primary cell that is capable of stable growth in vitro for many generations.

Recombinant host cells may be prokaryotic or eukaryotic, including bacteria such as E. coli, fungal cells such as yeast, mammalian cells such as cell lines of human, bovine, porcine, monkey and rodent origin, and insect cells such as Drosophila and silkworm derived cell lines. A recombinant host cell refers not only to the particular subject cell, but also to the progeny or potential progeny of such a cell. Because certain modifications can occur in succeeding generations due to either mutation or environmental influences, such progeny may not be identical to the parent cell, but are still intended to be included within the scope of the term.

Vectors of the present invention also include specifically designed expression systems that allow the shuttling of DNA between hosts, such as bacteria-yeast or bacteria-animal cells or bacteria-fungal cells or bacteria-invertebrate cells. Numerous cloning vectors are known to those skilled in the art and the selection of an appropriate cloning vector is within the purview of the artisan. For other suitable expression systems for both prokaryotic and eukaryotic cells see, e.g., chapters 16 and 17 of Maniatis et al., supra.

To obtain high level expression of a cloned gene or nucleic acid, such as a cDNA encoding a canine CCK1 receptor polypeptide, a canine CCK1 receptor sequence is preferably subcloned into an expression vector that contains a strong promoter to direct transcription, a transcription/translation terminator, and if for a nucleic acid encoding a protein, a ribosome binding site for translational initiation. Suitable bacterial promoters are known in the art and are described, e.g., by Sambrook et al., supra., and, CURRENT PROTOCOLS IN MOLECULAR BIOLOGY, Ausubel et al. (eds.), Greene Publishing Association and John Wiley Interscience, New York, 1989, 1992. Bacterial expression systems for expressing the CCK1 proteins disclosed in the present invention are available in, e.g., E. coli, Bacillus sp., and Salmonella (Palva et al., 1983, Gene, 22:229-235; Mosbach et al., 1983, Nature, 302:543-545). Kits for such expression systems are commercially available. Eukaryotic expression systems for mammalian cells, yeast, and insect cells are known in the art and are also commercially available. In exemplary embodiments, the eukaryotic expression vector is an adenoviral vector, an adeno-associated vector, or a retroviral vector.

A “promoter” is a regulatory sequence of DNA that is involved in the binding of RNA polymerase to initiate transcription of a gene. Promoters are often upstream (i.e., 5′ to) the transcription initiation site of the gene. A “gene” is a segment of DNA involved in producing a peptide, polypeptide, or protein, including the coding region, non-coding regions preceding (“5′UTR”) and following (“3′UTR”) coding region, as well as intervening non-coding sequences (“introns”) between individual coding segments (“exons”). “Coding” refers to the specification of particular amino acids or termination signals in three-base triplets (“codons”) of DNA or mRNA.

The promoter used to direct expression of a heterologous canine CCK1 receptor-encoding polynucleotide may be routinely selected to suit the particular application. The promoter is optionally positioned about the same distance from the heterologous transcription start site as it is from the transcription start site in its natural setting. As will be apparent to the artisan, however, some variation in this distance can be accommodated without loss of promoter function.

In addition to the promoter, the expression vector may contain a transcription unit or expression cassette that contains all the additional elements required for the expression of the canine CCK1 receptor-encoding polynucleotide in host cells. An exemplary expression cassette contains a promoter operably linked to the polynucleotide sequence encoding a canine CCK1 receptor polypeptide, and signals required for efficient polyadenylation of the transcript, ribosome binding sites, and translation termination. The polynucleotide sequence encoding a canine CCK1 receptor polypeptide may be linked to a cleavable signal peptide sequence to promote secretion of the encoded protein by the transfected cell. Exemplary signal peptides include the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

In exemplary embodiments, any of the vectors suitable for expression in eukaryotic or prokaryotic cells known in the art may be used. Exemplary bacterial expression vectors include plasmids such as pBR322-based plasmids, pSKF, pET23D, and fusion expression systems such as GST and LacZ. Examples of mammalian expression vectors include, e.g., pCDM8 (Seed, 1987, Nature, 329:840) and pMT2PC (Kaufinan et al., 1987, EMBO J., 6:187-195). Commercially available mammalian expression vectors which can be suitable for recombinant CCK1 expression include, for example, pMAMneo (Clontech), pcDNA3 (Invitrogen), pCiNeo (Promega), pMC1neo (Stratagene), pXT1 (Stratagene), pSG5 (Stratagene), EBO-pSV2-neo (ATCC 37593) pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

In yet other exemplary embodiments, the recombinant mammalian expression vector is capable of directing expression of the nucleic acid preferentially in a particular cell type (e.g., tissue-specific regulatory elements are used to express the nucleic acid). Various tissue-specific regulatory elements are known in the art. Examples of suitable tissue-specific promoters include the albumin promoter (liver-specific; Pinkert et al., 1987, Genes Dev., 1:268-277), lymphoid-specific promoters (Calame et al., 1988, Adv. Immunol., 43:235-275), such as promoters of T cell receptors (Winoto et al., 1989, EMBO J., 8:729-733), and immunoglobulins (Banerji et al., 1983, Cell 33:729-740; Queen et al., 1983, Cell, 33:741-748), neuron-specific promoters (e.g., the neurofilament promoter; Byme et al., 1989, Proc. Natl. Acad. Sci. U.S.A., 86:5473-5477), pancreas-specific promoters (Edlund et al., 1985, Science, 230:912-916), and mammary gland-specific promoters (e.g., milk whey promoter; U.S. Pat. No. 4,873,316 and European Patent Publication No. 264,166). Developmentally regulated promoters also include, for example, the marine hox promoters (Kessel et al., 1990, Science, 249:374-379) and the beta-fetoprotein promoter (Campes et al., 1989, Genes Dev., 3:537-546).

Epitope tags can also be added to recombinant proteins to provide convenient methods of isolation, e.g., c- myc, hemoglutinin (HA)-tag, 6-His tag, maltose binding protein, VSV-G tag, or anti-FLAG tag, and others known to those in the art.

Expression vectors containing regulatory elements from eukaryotic viruses can be used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo 5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedron promoter, or other promoters shown effective for expression in eukaryotic cells.

In exemplary embodiments, the pCiNeo expression vector is employed to introduce the canine CCK1 receptor polynucleotides of the present invention into host cells and to express them in transformed or transfected cells.

Some expression systems have markers that provide gene amplification, such as neomycin, thymidine kinase, hygromycin B phosphotransferase, and dihydrofolate reductase. Alternatively, high yield expression systems not involving gene amplification are also suitable, such as using a baculovirus vector in insect cells, with a sequence encoding a canine CCK1 receptor polypeptide under the direction of the polyhedrin promoter or other strong baculovirus promoters.

The elements that can be included in expression vectors also include a replicon that functions in E. coli; a gene encoding antibiotic resistance to permit selection of bacteria that harbor recombinant plasmids, and unique restriction sites in nonessential regions of the plasmid to allow insertion of eukaryotic sequences. The particular antibiotic resistance gene may be selected from the many resistance genes known in the art. The prokaryotic sequences may be chosen such that they do not interfere with the replication of the DNA in eukaryotic cells, if necessary or desired.

Known transfection methods may be used to produce bacterial, mammalian, yeast or insect cell lines that express large quantities of a canine CCK1 receptor polypeptide, which are then purified using standard techniques (see, e.g., Colley et al., 1989, J. Biol. Chem., 264:17619-17622; Guide to Protein Purification, in Methods in Enzymology, vol. 182, Deutscher, ed. (1990)). Transformation of eukaryotic and prokaryotic cells may be performed according to standard techniques (see, e.g., Morrison, 1977, J. Bact., 132:349-351; Clark-Curtiss & Curtiss, Methods in Enzymology 101:347-362, Wu et al., eds, (1983)).

Any of the known procedures suitable for introducing foreign nucleotide sequences into host cells may be used to introduce the expression vector. These include the use of reagents such as Superfect (Qiagen), liposomes, calcium phosphate transfection, polybrene, protoplast fusion, electroporation, microinjection, plasmid vectors, viral vectors, biolistic particle acceleration (the Gene Gun), or any other known methods for introducing cloned genomic DNA, cDNA, synthetic DNA or other foreign genetic material into a host cell (see, e.g., Sambrook et al., supra). The selected particular genetic engineering procedure used should be capable of successfully introducing at least one gene into the host cell capable of expressing a canine CCK1 receptor RNA, mRNA, cDNA, or gene.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) may be introduced into the host cells along with the gene of interest. Exemplary selectable markers include those which confer resistance to drugs, such as G418, puromycin, Geneticin, hygromycin and methotrexate. Cells stably transfected with the introduced nucleic acid can be identified by drug selection (e.g., cells that have incorporated the selectable marker gene will survive, while the other cells die).

A heterologous regulatory element can be inserted into a stable cell line or cloned microorganism, such that it is operatively linked with and activates expression of endogenous genes, using techniques such as targeted homologous recombination, e.g., as described in U.S. Pat. No. 5,272,071 and WIPO Publication No. WO 91/06667.

After the expression vector is introduced into the cells, the transfected cells are cultured under conditions favoring expression of the canine CCK1 receptor polypeptide, which is recovered from the culture using standard techniques identified below. Methods of culturing prokaryotic or eukaryotic cells are known and are taught, e.g., in Ausubel et al., supra, Sambrook et al., supra, and in Freshney, CULTURE OF ANIMAL CELLS, 3d ed., (1993), Wiley-Liss.

The isolated polypeptides of the present invention may be used in assay methods for identifying compounds that modulate a biological activity of a CCK1 receptor in test biological samples. Such assay methods are therefore useful for screening compounds as potential therapeutic agents for treating diseases or medical conditions mediated by CCK1 activity, such as CNS disorders, GI disorders, schizophrenia, Parkinson's disease, drug addiction, and feeding disorders. See, e.g., U.S. Pat. No. 6,169,173.

In preferred embodiments of such methods, canine CCK1 receptor polypeptides are isolated, e.g., from canine tissue such as brain, spleen, placenta, lung, liver, kidney, pancreas, prostate, testis, ovary, small intestine, colon, lymph node, and tonsils, or any other source of canine CCK1 receptor polypeptides. Bodily fluids such as blood, blood plasma, serum, seminal fluid, urine, or any other mammalian bodily fluid can also serve as sources of natural canine CCK1 receptor polypeptides. Cultured mammalian cell lines are still further exemplary sources of natural canine CCK1 receptor polypeptides.

In other embodiments, recombinant canine CCK1 polypeptides may be purified from any suitable bacterial or eukaryotic expression system, such as those described above. CCK1 proteins may be purified by standard techniques, including selective precipitation with such substances as ammonium sulfate; column chromatography; and immunopurification methods (see, e.g., Scopes, PROTEIN PURIFICATION: PRINCIPLES AND PRACTICE (1982); U.S. Pat. No. 4,673,641; Ausubel et al., supra; and Sambrook et al., supra).

A number of procedures can be employed when recombinant canine CCK1 receptor polypeptide is being purified. For example, proteins having established molecular adhesion properties can be reversibly fused to the canine CCK1 receptor polypeptide. With the appropriate ligand, a canine CCK1 receptor polypeptide can be selectively adsorbed to a purification column and then freed from the column in a substantially pure form. The fused protein is then removed by enzymatic activity. Canine CCK1 receptor proteins can also be purified using immunoaffinity columns.

Recombinant proteins may be expressed by transformed bacteria or eukaryotic cells in large amounts, preferably after promoter induction, but expression can be constitutive. Promoter induction with IPTG is one example of an inducible promoter system. Cells may be grown according to standard procedures in the art. Fresh or frozen cells may be used for isolation of protein.

Proteins expressed in bacteria may form insoluble aggregates (inclusion bodies). Several known protocols are suitable for purification of canine CCK1 receptor inclusion bodies. For example, purification of inclusion bodies may involve the extraction, separation and/or purification of inclusion bodies by disruption of bacterial cells, e.g., by incubation in a buffer of 50 mM TRIS/HCL pH 7.5, 50 mM NaCl, 5 mM MgC12, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria will be apparent to those of ordinary skill in the art (see, e.g., Sambrook et al., supra; Ausubel et al., supra).

If necessary or desired, the inclusion bodies may be solubilized, and the lysed cell suspension centrifuged to remove unwanted insoluble matter. Proteins that formed the inclusion bodies may be renatured by dilution or dialysis with a compatible buffer. Suitable solvents include urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of biologically active protein. Other suitable buffers are known in the art. Canine CCK1 receptor polypeptides are separated from other bacterial proteins by standard separation techniques, e.g., with Ni-NTA agarose resin.

Alternatively, CCK1 receptor polypeptides may be purified from bacteria periplasm. After lysis of the bacteria, when a canine CCK1 receptor protein is exported into the periplasm of the bacteria, the periplasmic fraction of the bacteria can be isolated by cold osmotic shock or another method known in the art. To isolate recombinant proteins from the periplasm, the bacterial cells may be centrifuged to form a pellet. The pellet may be resuspended in a buffer containing 20% sucrose. To lyse the cells, the bacteria may be centrifuged and the pellet resuspended in ice-cold 5 mM MgSO₄ and kept in an ice bath for approximately 10 minutes. The cell suspension may be centrifuged and the supernatant decanted and saved. The recombinant proteins present in the supernatant can be separated from the host proteins by standard separation techniques known in the art.

As an initial step, e.g., if a protein mixture is complex, an initial salt fractionation can be used to separate many of the unwanted host cell proteins (or proteins derived from the cell culture media) from the recombinant protein of interest. An exemplary salt is ammonium sulfate, which precipitates proteins by effectively reducing the amount of water in the protein mixture. Proteins then precipitate on the basis of their solubility. The more hydrophobic a protein is, the more likely it is to precipitate at lower ammonium sulfate concentrations. An exemplary isolation protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed to achieve the desired purity, e.g., through dialysis or diafiltration. Other known methods that rely on solubility of proteins, such as cold ethanol precipitation, can be used to fractionate complex protein mixtures.

In other examples, the molecular weight of a canine CCK1 receptor can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut-off greater than the molecular weight of the protein of interest. The recombinant protein will pass through the membrane into the filtrate. The filtrate can then be chromatographed.

Canine CCK1 receptor proteins can also be separated from other proteins on the basis of net surface charge, hydrophobicity, and affinity for heterologous molecules. In addition, antibodies raised against proteins can be conjugated to column matrices and the proteins immunopurified. It will be apparent to those of ordinary skill in the art that chromatographic techniques can be performed at any suitable scale and using equipment from many different manufacturers (e.g., Pharmacia Biotech).

Another general aspect of the invention relates to a method of identifying compounds that modulate the biological activity of a canine CCK1 receptor. Such modulators should be useful as therapeutic agents in treating a subject suffering from a disease or disorder related to the CCK1 receptor activity, such as CNS disorders (anxiety, schizophrenia, depression, Parkinson's disease, drug addiction, feeding/drinking disorders, pain or analgesia), metabolic disorders, proliferative disorders (e.g., pancreatic carcinogenesis), pancreatitis, pancreatic growth and enzyme secretion, disorders involving gastric antral motility and gastric emptying, relaxation of the sphincter of oddi and insulin secretion (see U.S. Pat. No. 6,169,173 and WIPO publication WO 93/16182). “Modulators” include both “inhibitors” and “activators”. “Inhibitors” refer to compounds that decrease, prevent, inactivate, desensitize or down-regulate canine CCK1 receptor expression or activity. “Activators” are compounds that increase, activate, facilitate, sensitize or up-regulate complex expression or activity.

The compound identification methods can be performed using conventional laboratory formats or in assays adapted for high throughput. High-throughput assays or screens (HTS) allow easy screening of multiple samples simultaneously or single samples rapidly, and can include the capacity for robotic manipulation. Another preferable feature of high-throughput assays is an assay design that is optimized to reduce reagent usage, or minimize the number of manipulations in order to achieve the analysis desired. Examples of assay formats include 96-well or 384-well plates, levitating droplets, microassays and “lab on a chip” microchannel chips used for liquid-handling experiments. Of course, as miniaturization of plastic molds and liquid-handling devices are advanced, or as improved assay devices are designed, greater numbers of samples will be able to be screened more efficiently using the inventive assay.

Candidate compounds for screening can be selected from numerous chemical classes, preferably from classes of organic compounds. Although candidate compounds can be macromolecules, preferably the candidate compounds are small-molecule organic compounds, i.e., those having a molecular weight of greater from 50 to 2500. Candidate compounds have one or more functional chemical groups necessary for structural interactions with polypeptides. Exemplary candidate compounds have at least an amine, carbonyl, hydroxyl or carboxyl group, preferably at least two such functional groups, and more preferably at least three such functional groups. The candidate compounds can comprise cyclic carbon or heterocyclic structural moieties and/or aromatic or polyaromatic structural moieties substituted with one or more of the above-exemplified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound is preferably a DNA or RNA molecule, although modified nucleic acids having non-natural bonds or subunits are also contemplated.

Candidate compounds may be obtained from a variety of sources, including libraries of synthetic or natural compounds. For example, numerous means are available for random and directed synthesis of a variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid-phase or solution-phase libraries; synthetic library methods requiring deconvolution; the “one-bead one-compound” library method; and synthetic library methods using affinity chromatography selection (see, e.g., Lam, 1997, Anti-Cancer Drug Des., 12:145). Alternatively, libraries of natural compounds in the form of bacterial, fungal, plant and animal extracts are available or may be routinely produced. Additionally, natural and synthetically produced libraries and compounds can be routinely modified through conventional chemical, physical, and biochemical means.

Furthermore, known pharmacological agents can be subjected to directed or random chemical modifications, such as acylation, alkylation, esterification, and amidification to produce structural analogs of the agents. Candidate compounds can be selected randomly or can be based on existing compounds that bind to and/or modulate the function or activity of a CCK receptor family member. Therefore, a source of candidate agents is known or screened libraries of molecules including activators or inhibitors of CCK1 receptors with similar structures to canine CCK1 receptor. The structures of such compounds may be changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions.

A variety of other reagents also can be included in the assay mixture. These include reagents such as salts, buffers, neutral proteins (e.g., albumin), and detergents that can be used to facilitate optimal protein-protein and/or protein-nucleic acid binding. Such a reagent can also reduce non-specific or background interactions of the reaction components. Other reagents that improve the efficiency of the assay, such as nuclease inhibitors, antimicrobial agents, and the like, can also be used.

Examples of methods for the synthesis of molecular libraries can be found in the art, for example in Zuckermann et al., 1994, J. Med. Chem., 37:2678. Libraries of compounds can be presented in solution (e.g., Houghten, 1992, Biotechniques, 13:412-421), or on beads (Lam, 1991, Nature, 354:82-84), chips (Fodor, 1993, Nature, 364:555-556), bacteria (U.S. Pat. No. 5,223,409), spores (U.S. Pat. No. 5,571,698), plasmids (Cull et al., 1992, Proc. Natl. Acad. Sci. U.S.A., 89:1865-1869) or phage (see e.g., Scott et al., 1990, Science: 249:386-390).

In one general embodiment, the invention provides a whole cell method to detect compound modulation of canine CCK1 receptor, comprising: (a) contacting a compound and a cell that contains biologically active CCK1 receptor material or a variant thereof; and (b) measuring change in the cell in response to modified receptor function by the compound. The amount of time for cellular contact with the compound may be empirically determined, for example, by running a time course with a reference CCK1 receptor modulator and measuring cellular changes as a function of time.

The measurement may be conducted by comparing a cell that has been exposed to a compound to an identical cell that has not been similarly exposed to the compound or, alternatively, to a cell that has been exposed to a reference compound (e.g., a known CCK1R modulator). Alternatively two cells, one containing the biologically active CCK1 receptor and a second cell identical to the first but lacking such receptor could be both be contacted with the same compound and compared for differences between the two cells. This technique is also useful in establishing the background noise of these assays. Artisans will, appreciate that these control mechanisms also allow easy selection of cellular changes that are responsive to modulation of the receptor.

The cellular changes suitable for the method of the present invention comprise directly measuring changes in the activity, function or quantity of canine CCK1 receptor, or by measuring downstream effects of the receptor function, for example by measuring secondary messenger concentrations or changes in transcription or by changes in protein levels of genes that are transcriptionally influenced by the receptor, or by measuring phenotypic changes in the cell. Preferred measurement means include changes in the quantity of canine CCK1 receptor protein, changes in the functional activity of the receptor, changes in the quantity of mRNA, changes in intracellular protein, changes in cell surface protein, or secreted protein, or changes in Ca+2, cAMP or GTP concentration. Changes in the levels of mRNA may be detected by reverse transcription polymerase chain reaction (RT-PCR) or by differential gene expression. Immunoaffinity, ligand affinity, or enzymatic measurement quantitates CCK1 induced changes in levels of specific proteins in host cells. Where the protein is an enzyme, the induction of protein may be monitored by cleavage of a fluorogenic or calorimetric substrate.

Preferred detection means for cell surface protein include flow cytometry or statistical cell imaging. In both techniques the protein of interest is localized at the cell surface, labeled with a specific fluorescent probe, and detected via the degree of cellular fluorescence. In flow cytometry, the cells are analyzed in a solution, whereas in cellular imaging techniques, a field of cells is compared for relative fluorescence.

The present invention is also directed to methods for screening for compounds that modulate the expression of DNA or RNA encoding canine CCK1 receptor as well as the function of the receptor protein in vivo. Compounds may modulate by increasing or attenuating the expression of DNA or RNA encoding the receptor, or the function of the receptor protein. Compounds that modulate the expression of DNA or RNA encoding the receptor or the function of the receptor protein may be detected by a variety of assays. The assay may be a simple “yes/no” assay to determine whether there is a change in expression or function. The assay may be made quantitatively by comparing the expression or function of a test sample with the levels of expression or function in a standard sample.

In another general embodiment, the invention relates to a method of identifying a compound that increases or decreases a biological activity of a canine cholecystokinin 1 receptor, comprising the steps of: (a) contacting (i) a test sample comprising a compound with (ii) an assay reagent comprising a biologically active canine CCK1 receptor polypeptide or a functional variant thereof and a cholecystokinin 1 receptor ligand; (b) determining the biological activity of the receptor after performing step (a); and (c) comparing the biological activity determined in step (b) with a control measurement obtained by contacting a control sample not containing the compound with the assay reagent. Preferably, the cholecystokinin 1 receptor ligand is a ligand selected from: sulfated CCK-8, desulfated CCK-8, desulfated ¹²⁵I-BH-CCK-8, sulfated ¹²⁵I-BH-CCK-8, L-364,718, YF476, and YM022.

A “ligand” or a “ligand component” refers to a chemical or peptidic moiety that binds to, or complexes with, a canine CCK1 receptor or variant thereof, such as sulfated CCK-8, desulfated CCK-8, desulfated ¹²⁵I-BH-CCK-8, sulfated ¹²⁵I-BH-CCK-8, L-364,718, L-365,260, YF476, YM022, and dexloxiglumide. Preferred ligands are high-affinity ligands, e.g., a ligand or ligand component that has a binding affinity constant, pK_{D} (negative log of K_{D}), for CCK1 receptor that is within the range of 10 and higher, or pK₁ (negative log of K₁) that is within the range of 7.9 and higher.

In a preferred embodiment, the assay reagent in the method is associated with a cell expressing the canine cholecystokinin 1 receptor on the cell surface. The term “cell” refers to at least one cell or a plurality of cells appropriate for the sensitivity of the detection method. Cells suitable for the present invention may be bacterial, but are preferably eukaryotic, such as yeast, insect, or mammalian. The cell can be a natural host cell for an endogenous canine cholecystokinin 1 receptor, preferably a recombinant host cell for a canine cholecystokinin 1 receptor, which expresses a high amount of a canine cholecystokinin 1 receptor on the cell surface.

In another preferred embodiment, the biological activity of the canine cholecystokinin 1 receptor or functional variant thereof can be measured by a second messenger response of the cell. For example, the biological activity of the complex can be measured by the signal transduction event triggered by activated canine cholecystokinin 1 receptor activation. This signal transduction event can be measured indirectly by means of measuring one or more changes in cellular physiology, such as cell morphology, migration, or chemotaxis, using one or more suitable methods known in the art. It can also be measured directly by measuring phosphorylation of proteins involved in the signal transduction pathway, for example, the phosphorylation of a GTP-binding protein (G protein). Methods are known in the art for measuring protein phosphorylation, for example, by using an ATP or GTP molecule that has been radiolabeled on the γ-phosphate.

A “second messenger response of a cell” refers to cellular response of the cell mediated through activation of a CCK1 receptor upon binding to, or complexing with, a ligand. It may include, e.g., signal transduction event or a change in intracellular concentration of a second messenger molecule, such as proton (pH), calcium, or cAMP.

The biological activity of a canine cholecystokinin 1 receptor material or variant can also be measured by the intracellular concentration of a second messenger molecule using any of a number of suitable techniques known in the art. For example, the pH change can be measured using a pH-sensitive dye, such as Acridine Orange. The calcium concentration can be measured via optical imaging of fluorescent indicators sensitive to Ca²⁺, such as fluo-3 (pentapotassium salt, cell-impermeant form; Molecular Probes) or fluo-3(AM) (an acetoxymethyl ester form of fluo-3, Teflabs) (see for example, Liu et al., 2001, J. Pharmacol. Exp. Ther., 299: 121-130) using a fluorometric imaging plate reader (FLIPR) or a confocal microscope. The cAMP concentration can be detected using a commercially available ELISA kit (FLASHPLATE cyclic AMP assay system (¹²⁵I, Cat. No: SMP001A, NEN; see also Shimomura et al., 2002, J. Biol. Chem., 277: 35826-35832), or via a reporter system wherein the expression of a reporter gene, such as beta-galactosidase, is under the control of a cAMP responsive element (cre) (Montminy et al., 1990, Trends. Neurosci., 3(5):184-188).

The test compound can be further characterized by comparing its effect on two cells, the first cell containing a biologically active canine cholecystokinin 1 receptor or functional variant thereof and the second one identical to the first, but lacking the active CCK1R or functional variant. This technique is also useful in establishing the background noise of these assays. One of ordinary skill in the art will appreciate that this control mechanism also allows ready selection of cellular changes that are responsive to modulation of functional canine cholecystokinin 1 receptor. Therefore, in a preferred embodiment, the screening method comprises the steps of: (a) contacting a first cell having a canine cholecystokinin 1 receptor (or functional variant) expressed on the cell surface with a cholecystokinin receptor ligand and with a test compound; (b) determining a second messenger response in the first cell to the test compound, and comparing it with that of a control wherein the first cell is only contacted with the cholecystokinin receptor ligand but not the test compound; (c) contacting a second cell with a cholecystokinin receptor ligand and with a test compound; wherein the second cell is otherwise identical to the first cell except that it does not express a canine cholecystokinin 1 receptor on the cell surface; (d) determining a second messenger response of the second cell to the test compound, and comparing the second messenger response with that of a control wherein the second cell is only contacted with the cholecystokinin receptor ligand but not the test compound; and (e) comparing the comparison result of (b) with that of (d).

There are a number of ways to obtain two cells that are otherwise identical except that one expresses a canine cholecystokinin 1 receptor on its cell surface and the other does not. In one embodiment, the first cell is a recombinant host cell for canine cholecystokinin 1 receptor that constitutively expresses canine cholecystokinin 1 receptor on its cell surface, and the second cell is the parent cell from which the canine cholecystokinin 1 receptor recombinant cell is constructed. In another embodiment, a recombinant host cell for the canine cholecystokinin 1 receptor is constructed such that its expression on the cell surface is under the control of an inducible promoter. The first cell is the recombinant cell grown under inducible conditions that allows the expression of canine cholecystokinin 1 receptor on its cell surface, and the second cell is the recombinant cell grown under non-inducible conditions that do not allow the expression of the canine cholecystokinin 1 receptor. In yet another embodiment, the first cell is a native host cell for canine cholecystokinin 1 receptor that expresses the polypeptide on its cell surface, and the second cell is a mutant cell derived from the native host, wherein the canine cholecystokinin 1 receptor gene has been inactivated through mutagenesis. Standard molecular biology methods can be used to construct a recombinant host cell for canine cholecystokinin 1 receptor, or to inactivate a canine cholecystokinin 1 receptor gene.

In another preferred embodiment, the present invention provides a method of identifying a compound that increases or decreases the activity of a receptor/ligand complex, comprising the steps of: (a) contacting an isolated membrane preparation comprising a CCK1 receptor with a ligand or an active fragment thereof with a test compound, and with a GTP molecule that has been labeled on the γ-phosphate; and (b) determining the amount of labeling bound to the membrane preparation; and (c) comparing the amount of labeling in (b) with that of a control wherein the membrane preparation is only contacted with the ligand or the active fragment thereof and the labeled GTP but not the test compound.

A variety of labels can be used to label the GTP molecule on the γ-phosphate, such as a fluorescent molecule or a radioactive isotope such as ³⁵S, ³²P, and the like.

In yet another embodiment, the present invention provides a method of identifying a compound that binds to a CCK1 receptor, comprising the steps of: (a) contacting a biologically active canine CCK1 receptor or variant thereof with a test compound, and with a labeled ligand or an active fragment thereof; (b) measuring the amount of the labeled ligand or the fragment thereof that binds to the receptor; and (c) comparing the measured amount of (b) with that of a control, wherein the receptor is only contacted with a labeled ligand or the fragment thereof, but not the test compound. The amount of the labeled ligand or fragment thereof that binds to the receptor can be measured by first separating the unbound labeled ligand or fragment from the receptor, and then measuring the amount of labeling that is associated with the receptor.

Separation of the receptor protein from unbound labeled ligand or fragments thereof can be accomplished in a variety of ways. Conveniently, the CCK1R material may be immobilized on a solid substrate, from which the unbound ligand can be easily separated. The solid substrate can be made of a variety of materials and in a variety of shapes, e.g., microtiter plate, microbead, dipstick, and resin particle. The substrate preferably is chosen to maximize signal-to-noise ratios, primarily to minimize background binding, as well as for ease of separation and cost. Separation can be effected by, for example, removing a bead or dipstick from a reservoir, emptying or diluting a reservoir such as a microtiter plate well, or rinsing a bead, particle, chromatographic column or filter with a wash solution or solvent. The separation step preferably includes multiple rinses or washes. For example, when the solid substrate is a microtiter plate, the wells can be washed several times with a washing solution, e.g., that includes those components of the incubation mixture that do not participate in specific bindings, such as salts, buffer, detergent, non-specific protein, etc. Where the solid substrate is a magnetic bead, the beads can be washed one or more times with a washing solution and isolated using a magnet.

CCK1R material can be immobilized on a solid substrate using a number of methods. In one embodiment, a fusion protein can be provided which adds a domain that allows the CCK1 proteins to be bound to a matrix. For example, glutathione-S-transferase fusion proteins or glutathione-S— transferase fusion proteins can be adsorbed onto glutathione sepharose beads (Sigma Chemical, St. Louis, Mo.) or glutathione derivatized microtiter plates, which are then combined with the test compound and the labeled ligand, and the mixture is incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or the labeled ligand to CCK1R material can be determined using standard techniques.

Other techniques for immobilizing proteins on matrices can also be used in the screening assays of the invention. For example, the canine CCKLR material can be immobilized utilizing conjugation of biotin and streptavidin. Biotinylated polypeptide can be prepared from biotin-NHS(N-hydroxy-succinimide) using techniques known in the art (e.g., biotinylation kit available from Pierce Chemicals, Rockford, Ill.), and immobilized in the wells of streptavidin-coated 96-well plates (Pierce Chemicals). Alternatively, antibodies reactive with the CCK1R but which do not interfere with binding of it to the ligand or test compound can be attached to the wells of the plate, and CCK1R then trapped in the wells by antibody conjugation.

A variety of labels can be used to label the ligand or fragments thereof, such as those that provide direct detection (e.g., radioactivity, luminescence, optical or electron density), or indirect detection (e.g., epitope tag such as the FLAG epitope, or enzyme tag such as horseradish peroxidase).

Other embodiments and features of the invention will become apparent by reference to the following illustrative examples.

### Example 1

### Cloning of the Canine CCK1 Receptor

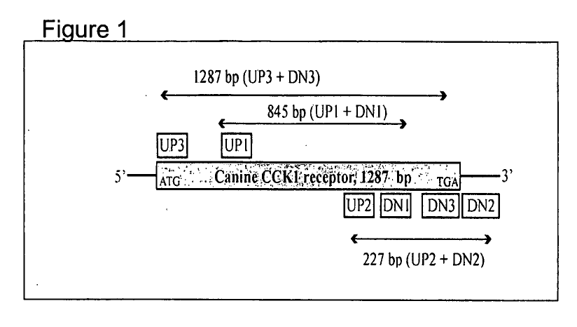

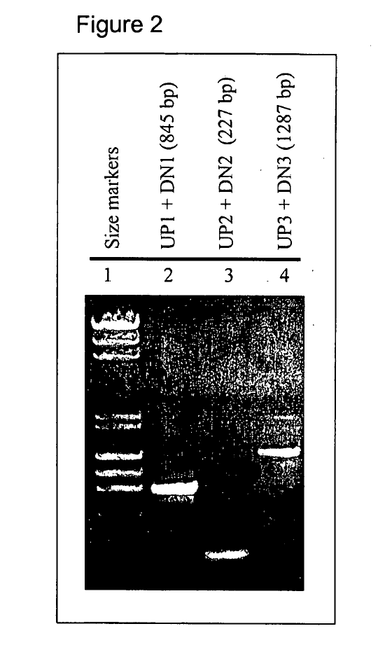

Cloning of a partial cDNA fragment of canine cholecystokinin 1 receptor (CCK1R) from canine gallbladder tissue by RT-PCR was performed by employing oligonucleotide primers complementary to the conserved region cDNA sequence between the human and rat CCK1 receptors (Accession Numbers 113605 and M88096). βactin primers were obtained from Maxim Biotech, Inc (San Francisco, Calif.). The upstream primer, UP1 (SEQ ID NO.:1), corresponding to base pairs 307 to 321, and the downstream primer, DN1 (SEQ ID NO.:2), corresponding to pase pairs 1152 to 1172 (FIG. 1, with sequences shown in Table 1), amplified an 845-bp PCR product corresponding to the majority of the middle region of the canine CCK1 cDNA (FIG. 2).

TABLE 1

Sequences and species specificity of
oligonucleotide primers

| | | |
|---|---|---|
| Primer | Sequence | Species |

| | | |
|---|---|---|
| UP1 | 5′-CTGCTCAAGGATTTCATCTTCGG-3′ | human/rat |
| | (SEQ ID NO.:1) | |
| DN1 | 5′-GGGAAGGTGGCCATGAAGCC-3′ | human/rat |
| | (SEQ ID NO.:2) | |
| UP2 | 5′-CATTTCCTTCATCCTCCTGCTGTCC | canine |
| | T-3′ | |
| | (SEQ ID NO.:3) | |
| DN2 | 5′-CGCTCAGGGGCCCGGGGCCGA-3′ | canine |
| | (SEQ ID NO.:4) | |
| UP3(EcoRI) | 5′-AACGTTGGAATTCGCCACCATGGAGG | canine |
| | TGGCCGACAGCCT-3′ | |
| | (SEQ ID NO.:5) | |
| DN3(NotI) | 5′-AACGTTGCGGCCGCTCAGGGGCCCGG | canine |
| | GGCCGAGGCGC-3′ | |
| | (SEQ ID NO.:6) | |
| βactin(UP) | 5′-CATGGGCCAGAAGGACTCCTAC-3′ | canine |
| | (SEQ ID NO.:9) | |
| βactin(DN) | 5′-CACGCTCCGTGAGGATGTTC-3′ | canine |
| | (SEQ ID NO.:10) | |

Reverse transcription (RT) reactions on canine tc-RNA were performed in a 20μl reaction mixture containing 10 mM Tris-HCl (pH 8.4), 50 mM KCl, 5 mM MgCl₂, 500 μM dNTP, 1.25 μM of Oligo(dT) primer, 5 μg tc-RNA, 40 units of Rnase inhibitor and 50 units of Reverse TranscriptaseII (Invitrogen, Carlsbad, Calif.). The cDNA (1 μl) samples were immediately used in PCR with the addition of 45 μl of Supermix (Invitrogen) containing 2.2 units of Taq DNA polymerase (a mixture of recombinant Taq DNA polymerase and DNA polymerase from pyrococus species GB-D) in 66 mM Tris-SO₄ (pH 9.1 at 25° C.), 19.8 mM (NH₄)₂SO₄, 2.2 mM MgSO₄, 229 μM dGTP, 220 μM dATP, 220 μM dTTP, 220 μM dCTP, with stabilizers and 20 μM of sense and antisense primers. The RT reactions were performed under the following conditions: 90 min at 42° C., 10 min at 70° C. followed by 20 min at 37° C. in the presence of 2 units of RnaseH. The cDNA fragments were amplified by PCR under the following conditions: 30 s at 94° C. for 1 cycle, followed by 94° C. for 30 s, 30 s at 60° C., 72° C. for 3 min for 30 cycles. The 845-bp fragment (FIG. 2) was confirmed by sequencing to be the canine CCK1 receptor that matched the publicly available canine genomic DNA sequence.

From the canine CCK1 receptor sequence of the 845-bp PCR fragment, a 26-mer sense primer, UP2 (SEQ ID NO.: 3), was synthesized (FIG. 1 and Table 1); In addition, a 21-mer oligonucleotide antisense primer, DN2 (SEQ ID NO.: 4), was synthesized based on the canine whole genome shotgun (WGS) sequence overlapping with the 3′ end of the 845-bp partial cDNA. The canine WGS sequences were downloaded from NCBI (at ftp://ftp.ncbi.nih.gov/pub/TraceDB/canis-familiaris/), and sequences sharing homology with canine CCK1 partial cDNA and human CCK1 3′ end sequence were assembled in Vector NTI suites (Infomax, Calif.). Primers were designed with the consensus sequence. RT-PCR was performed on tc-RNA isolated from canine gallbladder using primers UP2 and DN2 (which contained the stop codon) to isolate the 3′ end (227 bp, see FIG. 2). This enabled the design of primer DN3 (Not1), which was used in conjunction with UP3(EcoRI) to amplify the full-length canine CCK1 receptor cDNA (1287 bp; see FIG. 2 for gel images of amplified PCR products). The PCR conditions were the same as described above. The cDNA fragments were sequenced and the start and stop codon determined.

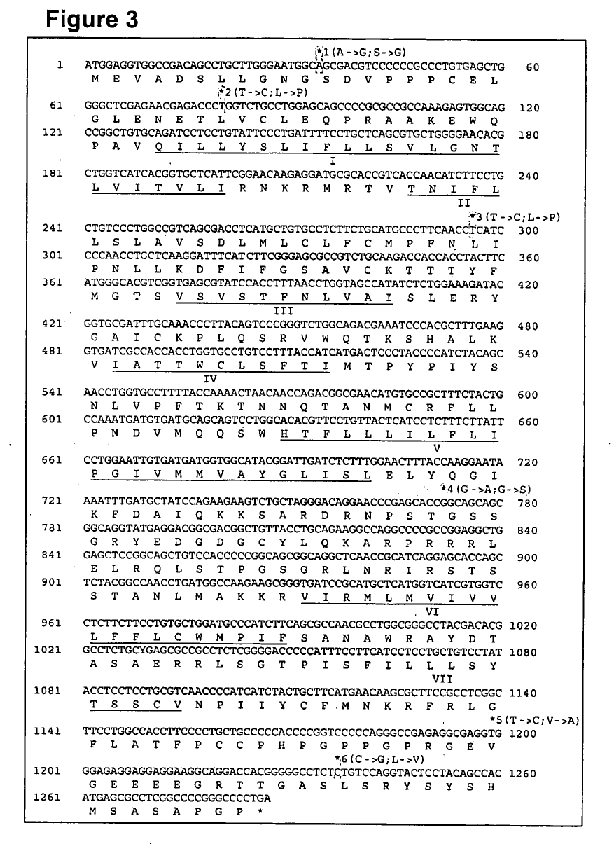

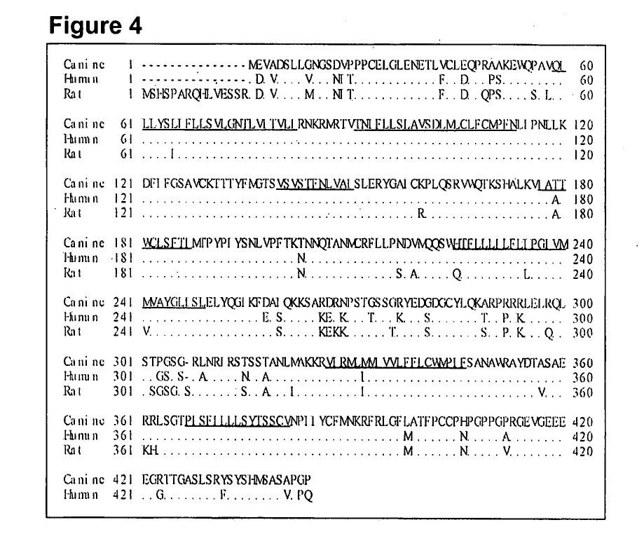

In addition, the complete coding region of the canine CCK1 receptor was amplified by RT-PCR from total RNA isolated from canine colon and a CNS library. The cDNA amplification product was sequenced (FIG. 3) and found to be 89% identical to the human and 85% identical to the rat CCK1 receptor (FIG. 4). The ORF encodes a 428 amino-acid protein, which shares 85% and 84% identity with the human and rat CCK1 receptors, respectively.

### Sequencing of Canine CCK1 PCR Product and Identification of Canine CCK1 Variants

The cDNA PCR product was subcloned into the mammalian cloning vector pCi Neo (Promega). Recombinant double-stranded plasmids served as templates for cycle sequencing with T7 forward and T3 reverse primers and fluorescence-based dideoxynucleotides, using the dideoxy-terminator cycle sequencing kit (Perkin Elmer, Inc). Sequences were determined by use of a DNA Sequencer (ABI Model 373, Applied Biosystems, Foster City, Calif.) and compared to the sequence described by Kirkness and co-workers (Kirkness et al., 2003, Science, 301:1898-1903). Sequences were validated by sequencing RT-PCR products from three separate RT-PCR reactions.

The sequencing of the canine CCK1 receptor also identified two additional variants of the canine CCK1 receptor (see FIG. 3 for location of nucleotide alterations, each denoted with an asterisk). These were identified in three independent PCR reactions from three separate transformed colonies all conducted using high-fidelity Taq polymerase. These additional variants were termed variant #1 (3 a.a. changes compared to wild-type) and variant #2 (6 a.a. changes compared to wild-type).

### Cloning of Canine Cholecystokinin cDNA into Expression Vectors

The full-length canine CCK1 receptor cDNA of the originally-identified receptor sequence was subcloned and inserted into a mammalian expression vector pCiNeo (Promega, San Luis Obispo, Calif.) for expression studies. Two 37-49 bp chimeric oligonucleotide primers were synthesized to facilitate the subcloning. The chimeric upstream primer (UP3(EcoR1)) includes two adjacent sequences (6 random bases followed by 6 bps of EcoR1 sequence), a 6-bp Kozak sequence and a 20-bp sequence complementary to the canine CCK1 receptor cDNA sequence (1-20 bp). The chimeric downstream primer (DN3(Not1)) includes six random base pairs followed by Not1 restriction site and twenty base pairs complementary to the canine CCK1 receptor cDNA sequence 1270-1290 (Table 1). PCR with the above two chimeric primers resulted in a 1296-bp product. The purified PCR products and the expression vector were digested with EcoR1 and Not1, ligated and transformed into DH5 alpha cells (Invitrogen, San Diego, Calif.). The transformed cells were then screened for carbenicillin (Gemini, Woodland, Calif.) resistant (50 μg/ml) recombinant plasmids.

In order to investigate the other two variant clones that were obtained from the same gallbladder tissue, plasmid DNAs containing the respective variant canine CCK1 receptor cDNAs and the wild-type cDNA were transiently transfected into HEK-293 cells using lipofectamine 200 transfection reagent and Opti-MEM medium (Invitrogen, Carlsbad, Calif.). Cells were harvested at 48-72 hours after transfection and the pellets were frozen at −80° C.

### Example 2

### Analysis of Tissue Distribution of the Canine CCK1 Receptor

Semi-quantitative RT-PCR analysis of canine CCK1 receptor expression was performed in order to determine the tissue distribution of canine CCK1. Five □g of total cellular RNA was reverse transcribed using oligo dT primer following manufacturers instructions (TaqMan RT, Applied Biosystems, Foster City, Calif., USA). To conduct real-time PCR, 5 μl of cDNA was incubated with 25 μl SYBR Green (Applied Biosystems), 3 μl of 5 mM of forward and reverse primers (forward primer (SEQ ID NO.:7):5′-CATCTACAGCAACCTGGTGC-3′, reverse primer (SEQ ID NO.:8): 5′-GTGGACAGCTGCCGGAGCTC-3′) and 14 μl water to give a total volume of 50 μl. The PCR reaction was conducted using the iCycler™ real time PCR machine (Bio-rad, Hercules, Calif., USA) and the cycle times were 1×95° C. 4 min, 35×1 min 60° C., 1 min 72° C., 1 min 94° C. All samples were assayed in triplicate and samples where no reverse transcriptase had been included were used as control. For each sample, the level of transcript input was estimated by normalizing to a βactin control.

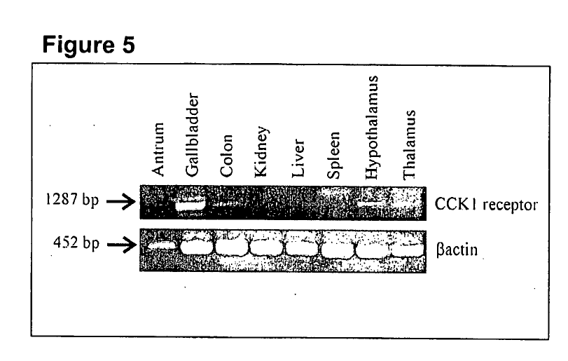

RT-PCR of the canine CCK1 receptor indicated expression of this receptor in gallbladder, colon, hypothalamus and thalamus but not in kidney, liver, spleen and gastric antrum (FIG. 5). From these results it appeared that the highest level of expression was seen in the canine gallbladder tissue.

### Example 3

### Comparison of the Affinity Values Estimated at the Cloned Canine and Human CCK1 Receptor and at the Canine CCK2 Receptor

Compounds and materials used in the experiments described in this and in the following examples include [¹²⁵I]-BH-CCK-8S (specific activity ˜2200 Ci.mmol⁻¹), which comprises a sulfated eight amino-acid cholecystokinin peptide that has been radioiodinated by Bolton-Hunter conjugation (see, e.g., Bolton et al., 1973, Biochem. J, 133(3):529-539), was supplied by Amersham, Buckinghamshire, UK. 2-NAP (2-naphthalenesulponyl 1-aspartyl-(2-phenylethyl)amide), YF476 ((R)-1-[2,3-dihydro-2-oxo-1-pivaloylmethyl-5-(2′-pyridyl)-1H-1,4-benzodiazepin-3-yl]-3-(3-methyl-phenyl)urea), YM022 (1-[(R)-2,3-dihydro-1-(2-methylphenacyl)-2-oxo-5-phenyl-1H-1,4-benzodiazepin-3-yl]-3-(3-methylphenyl)urea), L-364,718 (3S(−)—N-(2,3-dihydro-1-methyl-2-oxo-5-phenyl-1H-1,4-benzodiazepine-3-yl-1H-indole-2-carboxamide), L-365,260 (3R(+)—N-(2,3-dihydro-1-methyl-2-oxo-5-phenyl-1H-1,4-benzodiazepin-3-yl)-N′-(3-methylphenyl)urea), dexloxiglumide (R)-4-(3,4-Dichloro-benzoylamino)-4-[(3-methoxy-propyl)-pentyl-carbamoyl]-butyric acid were synthesised in house or supplied by the James Black Foundation Ltd, London. All compounds were dissolved in dimethyl formamide to give stock concentrations of 1 mM and further dilutions were made in 50 mM Tris-HCl buffer.

The plasmids described above were used for stable transfection into CHO-K cells (Chinese Hamster Ovary) (American Type Culture Collection, Rockville, Md.) using the Effectene transfection method (Qiagen, Chatsworth, Calif.) with 2 μg plasmid for each 100 mm² culture dish. These cells were maintained in Ham's F12 selection medium with 10% fetal bovine serum, 2 mM L-glutamine, penicillin (50 U ml⁻¹), streptomycin (50 mg ml⁻¹) and Geneticin (0.7 mg ml⁻¹) (Invitrogen) at 37° C. in a humidified incubator under an atmosphere containing 5% CO₂. Media was changed every other day. Isolated Geneticin resistant colonies were picked from the 100 mm² dishes and grown to confluence in 6 well cluster dishes. 24 individual stable clones were used in a Fluorometric Imaging Plate Reader (FLIPR) assay with CCK-8S (0.01 nM-1 μM) to select a clone with a good signal-to-noise window for use in further experiments.

For radioligand binding studies, the cells were harvested by cell scraping and resulting pellets immediately frozen at −80° C. (approximately 50×10⁶ cells/pellet). Frozen cell pellets were defrosted on ice in 15 ml of assay buffer (composition; 10 mM HEPES, 130 mM NaCl, 4.7 mM KCl, 5 mM MgCl2, bacitracin 0.089; pH 7.2 at 21±3° C.) and then homogenized (setting 10, 7×3 s; Polytron; Brinkmann Instruments). The homogenate was centrifuged (800×g for 10 min) and the pellet discarded. The supernatant was re-centrifuged (39,800×g for 25 min) and the final pellet re-suspended in 20 ml assay buffer (cell concentration: 25×10⁵ cells ml⁻¹). Protein concentration was determined using BCA Protein Assay Kit (Pierce, Rockford, Ill.). All binding assays were conducted in 96 well Multiscreen GF/B filter plates (Millipore, Billerica, Mass., USA) that were pre-soaked in assay buffer for 1 h. For competition studies, cell membranes (45 μl) were incubated with 60 μM [¹²⁵I]-BH-CCK-8S (50 μl) in the presence of competing ligand (15 μl) for 90 min (total volume of 150 μl). Nonspecific binding was determined by inclusion of 10 μM 2-NAP (a CCK1 receptor selective antagonist; see, e.g., Hull et al., 1993, Br. J. Pharmacol., 108:734-740). All radioligand binding studies were conducted in the presence of the CCK2 receptor-selective antagonist PD-134,308 at a concentration estimated to occupy 99% of human CCK2 receptors (0.3 μM; se, e.g., Hughes et al., 1990, Proc. Natl. Acad. Sci. U.S.A., 87(17):6728-6732; Hunter et al., 1993, Mol. Pharmacol., 43:595-602). The bound radioactivity was separated by filtration using a Multiscreen Resist manifold (Millipore, Billericay, Mass., USA). The filters were washed three times with ice-cold PBS (pH 7.5) and radioactivity retained on the filters was measured by liquid scintillation counting using a TopCount (Packard BioScience, Boston, Mass.). All experiments were performed in triplicate.

The individual competition curve data were expressed as the percentage in the decrease of specific [¹²⁵I]-BH-CCK-8S binding (B) within each experiment. These data were then analysed using a four-parameter logistic (eqn. 1; GraphPad Prism 3.02) with the upper (αₘₐₓ) and lower (αₘᵢₙ) asymptotes weighted to 100% and 0% by including these values two log units above and below the lowest and highest concentrations of competitor, respectively. Equilibrium dissociation constant (K₁) values were assumed to be equal to the IC₅₀ values obtained from the logistic curve fit as the radiolabel concentrations used in the assays were always below the K_{D} of the radiolabel. The equilibrium dissociation constants (K₁) values were calculated from the midpoint locations (IC₅₀) following Cheng & Prusoff, eqn. 2 (Cheng et al., 1973, Biochem. Pharmacol., 22:3099-3108):

$$\begin{matrix}B = \frac{{α}_{min} + ( {α}_{max} - {α}_{min} )}{1 + {10}^{( ( log {IC}_{50} - [ L ] ) \cdot {n}_{ll} )}} & ( 1 ) \\ {K}_{l} = \frac{{IC}_{50}}{1 + \frac{[ L ]}{{K}_{D}}} & ( 2 )\end{matrix}$$

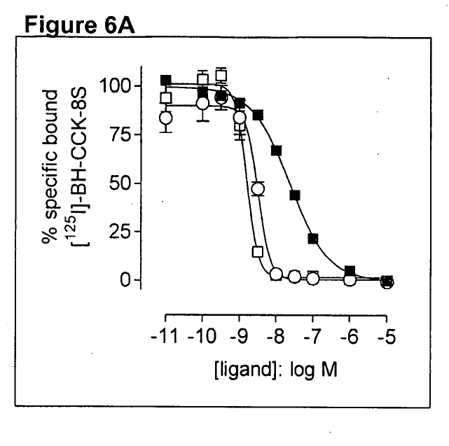

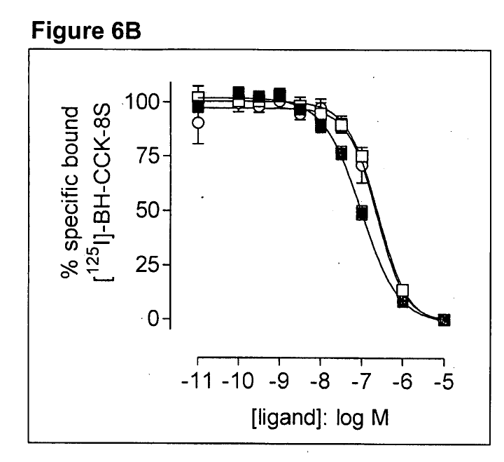

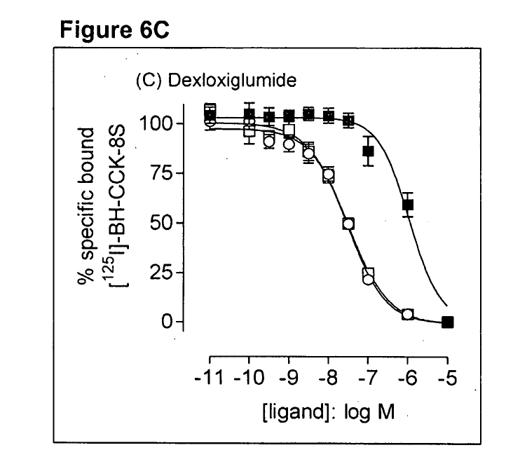

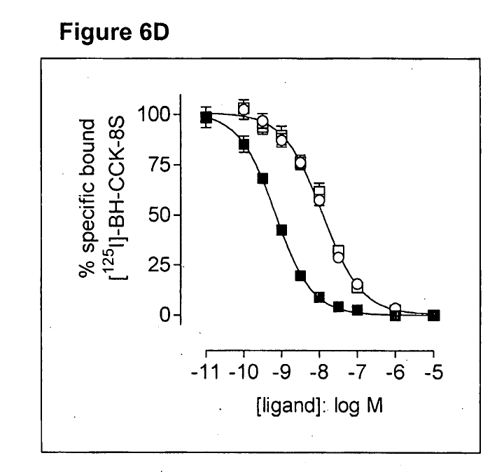

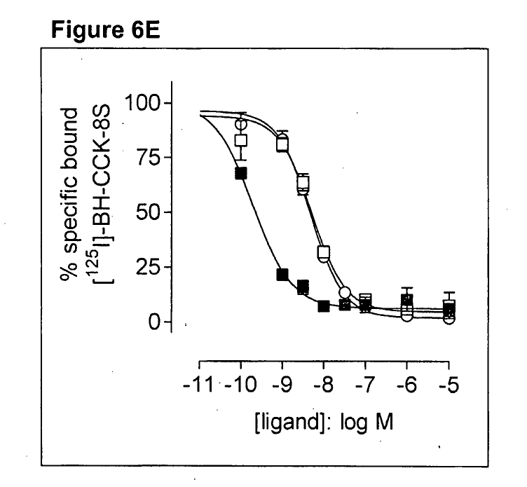

Competition-inhibition studies were conducted on the canine and human CCK1 receptor using a cell-protein concentration within the linear range of the cell number curve (80 μg/ml, cell number curves not shown). All CCK receptor selective ligands produced a concentration-dependent decrease in the amount of specific bound [¹²⁵I]-BH-CCK-8S (FIGS. 6A-6E). There were no significant differences in the affinity values estimated for these compounds at the human and canine CCK1 receptor (Table 2). The compounds L-365,260 and YF476 and YM022 expressed ˜2.6, ˜12- and 27-fold higher affinity, respectively at the canine CCK2 receptor compared to the canine CCK1 receptor. In comparison, L-364,718 and dexloxiglumide had a 16- and 93-fold higher affinity at the canine CCK1 receptor. The analysis of the competition-inhibition data of L-364,718 revealed that the Hill slope for this compound was significantly greater than unity in all assays conducted at the human and canine CCK1 receptor (see Table 2).

TABLE 2
Affinity values (−log equilibrium dissociation constants)
with corresponding Hill slopes (n_{H}) estimated for CCKR antagonists
at the cloned human and canine CCK1R and at the canine CCK2R
using [¹²⁵I]-BH-CCK-8S as radiolabel (n = 3, conducted in triplicate, values ± s.e. mean)

| | Canine CCK1 | | Human CCK1 | | Canine CCK2 | |
|---|---|---|---|---|---|---|
| | pK_{I} | n_{H} | pK_{I} | n_{H} | pK_{I} | n_{H} |
| L-364,718 | 8.82 ± 0.08 | 2.54 ± 0.19 | 8.71 ± 0.09 | 2.86 ± 0.04 | 7.62 ± 0.04 | 0.87 ± 0.06 |
| L-365,260 | 6.61 ± 0.05 | 1.17 ± 0.09 | 6.61 ± 0.06 | 1.32 ± 0.14 | 7.03 ± 0.15 | 0.95 ± 0.08 |
| YF476 | 7.91 ± 0.15 | 0.88 ± 0.08 | 7.87 ± 0.08 | 0.95 ± 0.07 | 8.98 ± 0.12 | 0.91 ± 0.10 |
| YM022 | 8.28 ± 0.06 | 1.09 ± 0.13 | 8.11 ± 0.05 | 1.15 ± 0.14 | 9.71 ± 0.06 | 0.92 ± 0.10 |
| dexloxiglumide | 7.53 ± 0.11 | 0.83 ± 0.13 | 7.53 ± 0.07 | 0.91 ± 0.18 | 5.56 ± 0.05 | 0.71 ± 0.09 |

### Example 4

### Comparison of the Affinity Values Estimated at the Wild-Type and Variant Canine CCK1 Receptors

Two additional canine CCK1 receptor variants, which were obtained from the same gall bladder tissue, were identified during the cloning of the canine CCK1 receptor. These variant forms of the receptor protein had three (variant #1, SEQ ID NO.:15) and six (variant #2, SEQ ID NO.:16) amino-acid differences when compared to the published genomic canine sequence. In order to investigate these variants, plasmid DNAs containing the respective variant canine CCK1 receptor cDNAs and the wild type cDNA were transiently transfected into HEK-293 cells using Lipofectamine 200 transfection reagent and Opti-MEM medium (Invitrogen). Cells were harvested at 48-72 hours after transfection and the pellets were frozen at −80° C. The cells were then harvested, assayed, and the data analyzed as described above.

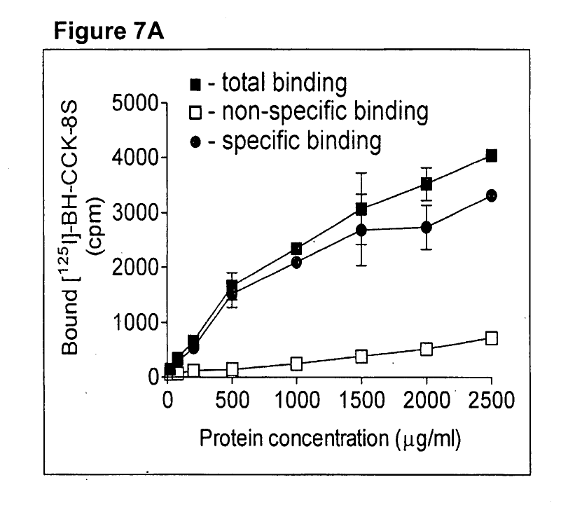

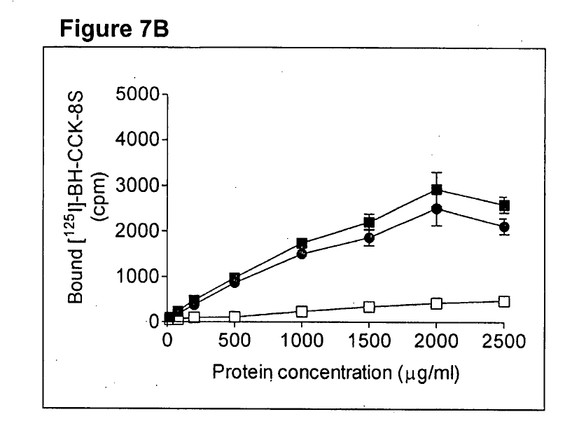

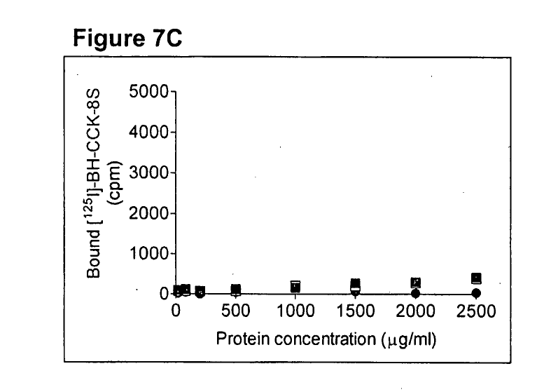

No specific binding was detected for variant #2 up to a protein concentration of 2500 μg/ml (FIG. 7C). Conversely, the binding of [¹²⁵I]-BH-CCK-8S to variant #1 and the control wild-type canine CCK1R protein (SEQ ID NO.:14) increased with increasing cell number (FIGS. 7A-7B). The competition-inhibition studies demonstrated that all CCK-receptor selective ligands investigated produced a concentration-dependent displacement of bound [¹²⁵I]-BH-CCK-8S to both the wild type and variant #1 canine CCK1 receptor (Table 3). There was no significant difference in the affinity values of the CCK receptor selective ligands between the wild type and variant #1 canine CCK1 receptors.

TABLE 3
Affinity values (−log equilibrium dissociation constants)
with corresponding Hill slopes (n_{H}) values estimated for CCKR
antagonists at the control and variant canine CCK1 receptors using
[¹²⁵I]-BH-CCK-8S as radiolabel (n = 3, conducted in
triplicate, values ± s.e. mean)

| | Canine CCK1: Wild-type | | | |
|---|---|---|---|---|
| | (transiently | | Canine CCK1: Variant #1 | |
| | transfected in | | (transiently transfected in | |
| | HEK cells) | | HEK cells) | |
| | pK_{I} | n_{H} | pK_{I} | n_{H} |
| L-364,718 | 8.42 ± 0.07 | 3.85 ± 2.66 | 8.60 ± 0.07 | 1.78 ± 0.50 |
| L-365,260 | 6.66 ± 0.19 | 0.96 ± 0.32 | 6.81 ± 0.15 | 1.08 ± 0.33 |
| YF476 | 7.92 ± 0.23 | 0.60 ± 0.22 | 7.82 ± 0.14 | 1.14 ± 0.37 |
| YM022 | 7.96 ± 0.11 | 1.17 ± 0.30 | 8.08 ± 0.10 | 1.39 ± 0.39 |
| dexloxiglumide | 7.78 ± 0.12 | 1.33 ± 0.43 | 7.78 ± 0.10 | 0.90 ± 0.17 |

### Example 5

### Comparison of the Saturation Binding Data for [¹²⁵]-BH-CCK-8S at the Human and Canine CCK1 Receptors

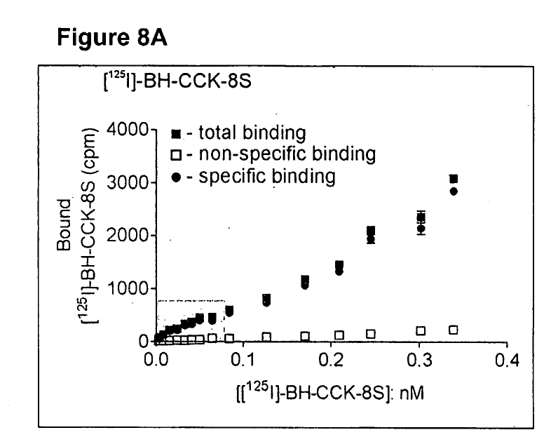

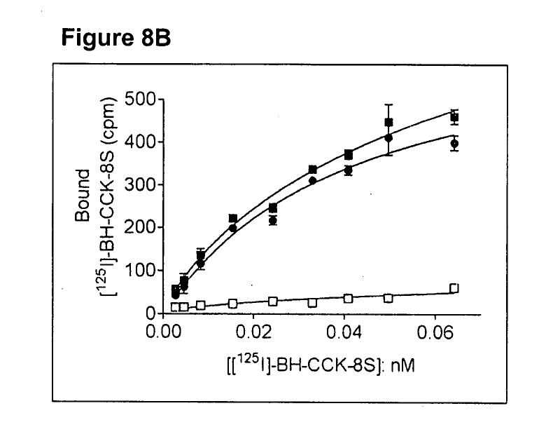

The plasmids containing the respective canine CCK1 cDNAs described above were transfected into HEK-293 cells, harvested, assayed, and the data analyzed as described above. The binding of [¹²⁵I]-BH-CCK-8S increased with increasing concentration of radioligand at the wild-type (FIGS. 8A-8B) and variant #1 canine CCK1 receptors and human CCK1 receptor. However, no specific binding was measured at the canine CCK1 variant #2 receptor, with [¹²⁵I]-BH-CCK-8S concentrations ranging from 2 pM to 0.3 nM (using 500 μg/ml protein). For the CCK1 receptor saturation experiments, the binding isotherm of [¹²⁵I]-BH-CCK-8S did not reach a maximum over the concentration range used and the data appeared biphasic. This is illustrated with the data obtained using the wild-type canine CCK1 receptor in FIG. 8A. An estimate of the affinity of [¹²⁵I]-BH-CCK-8S was obtained by analyzing the data obtained over the first phase of specific binding corresponding to a concentration range of 2 μM to 0.08 μM. From these data, the estimated pK_{D} values for [¹²⁵I]-BH-CCK-8S at the canine (wild-type and variant #1) and human CCK1 receptors were not significantly different (canine CCK1 receptor stably transfected in CHO cells pK_{D}=10.46±0.09; canine CCK1 receptor transiently transfected in HEK cells pK_{D}=10.30±0.02; canine variant #1 CCK1 receptor pK_{D}=10.24±0.08; human CCK1 receptor pK_{D}=10.26±0.05, n=3, conducted in triplicate).

### Example 6

### Quantitation of the Wild-Type, Variant #1 and Variant #2 CCK1 Receptor in the Transiently Transfected Cell Lines

The amount of canine CCK1 receptor RNA, relative to βactin control, was determined by real time PCR in the HEK cells transiently transfected with the wild-type, variant #1, and variant #2 canine CCK1 receptors (SEQ ID NOs.: 11, 12, and 13), respectively, as described above. The expression of the variant #2 CCK1 receptor was significantly lower than the wild-type and variant#1 CCK1 receptor (expression levels relative to βactin control: wild-type CCK1=17.3±2.8, variant #1 CCK1 receptor=10.4±2.9, variant #2 CCK1 receptor=3.3±0.5, untransfected HEK cells had no detectable expression).

### DISCUSSION

CCK1 receptors have been cloned from a number of species including rat, human, guinea-pig, rabbit, mouse and, most recently, cynomolgus monkey (Wank et al., 1992, Proc. Natl. Acad. Sci. U.S.A., 89:3125-3129; Ulrich et al., 1993, Biochem. Biophys. Res. Commun., 193:204-211; de Weerth et al., 1993, Am. J. Physiol., 265:G1116-G1121; and Reuben et al., 1994, Biochim. Biophys. Acta., 1219:321-327; Ghanekar et al., 1997, Pharmacol. Exp. Ther., 282:1206-1212; and Holicky et al., 2001, Am. J. Physiol. Gastrointest. Liver Physiol., 281:G507-G514, respectively). Many of the actions of CCK and gastrin have been investigated in canine gastrointestinal tissue because of the similarity of the canine GI tract with the human gut. However, the interpretation of these data has been limited by the lack of selectivity of the reference antagonists L-365,260 and L-364,718 at the canine CCK2 receptor and also by the absence of affinity values for these compounds at a homogenous population of canine CCK1 receptors. Consequently, and in view of the reports of significant differences in the pharmacology of synthetic ligands between the canine and human CCK2 receptors (Beinborn et al., 1993, Nature, 362:348-350), we cloned, expressed and performed a pharmacological analysis of the canine CCK1 receptor.

The canine CCK1 receptor was identified through the use of primers designed to interact with conserved regions of the human and rat CCK1 receptor. These primers amplified a large section of the canine CCK1 receptor from gallbladder tissue (845 bp). From this, additional primers were designed which, when used in conjunction with primers identified from the canine genomic sequence, amplified the full length of the canine CCK1 receptor. This sequence was highly homologous with the CCK1 receptor from other species (85% amino-acid identity with the rat and 89% amino-acid identity with the human CCK1 receptor). In addition, to the wild-type canine CCK1 receptor, we also identified two further forms of the receptor (variant #1 and #2), which contained 3 and 6 amino-acid mutations; respectively. These variants were identified in three separate PCR reactions conducted using high-fidelity taq polymerase from distinct colonies of cells and, therefore, it seems unlikely that these arose from PCR-induced mutations. However, because these experiments were performed on RNA obtained from a single animal additional sequencing of this receptor across a broader population is required to ascertain if these polymorphisms can be considered single nucleotide polymorphisms.

The pharmacology of the cloned canine CCK1 receptors was investigated using a number of previously characterized, structurally diverse, CCK-receptor selective antagonists. In addition, the canine CCK2 and human CCK1 receptors were included within each experiment so that a direct comparison of the antagonist affinity values could be made. No significant differences in the affinity of L-364,718, L-365,260, YF476, YM022 and dexloxiglumide were observed between the canine and human CCK1 receptor. Therefore, in contrast to the pharmacology of the canine and human CCK2 receptors (Beinborn et al., 1993, Nature, 362:348-350), no differences in the rank potency order of L-364,718 or L-365,260 were observed. Previously, the affinities of L-365,260, L-364,718 and YM022 have been investigated in radioligand binding studies conducted on canine small intestine circular muscle using L-364,718 as radioligand (Mao et al., 1995, Peptides, 16:1025-1029). Consistent with our results, Mao and co-workers demonstrated that L-364,718 expressed a higher affinity than L-365,260 at putative CCK1 receptor binding sites labeled with [³H]-L-364,718, however, they reported no inhibitory effect of YM022 at the same binding sites expressed in the canine intestine. This is in contrast to the results described above, which demonstrated that YM022 expressed a relatively high affinity for both human and canine CCK1 receptors. Similarly, we have previously demonstrated that the enantiomer of YM022, YF476, expressed a high affinity at CCK1 receptors in human gallbladder tissue (Morton et al., 2002). It is unlikely that the high affinity of YF476 and YM022 shown here results from any displacement of [¹²⁵I]-BH-CCK-8S from CCK2 receptors because of the following: (i) the Hill slopes for YF476 and YM022 were not significantly different from unity consistent with displacement from a single site; (ii) the radioligand binding studies were conducted in the presence of a high, but CCK2 receptor selective, concentration of the ligand PD-134,308 (3 μM), and (iii) the value obtained for L-365,260 also used in this study was consistent with its displacement from CCK1 receptors. Therefore, in contrast to its reported pK₁ value of ˜6.5 at guinea-pig pancreatic CCK1 receptors (Takinami et al., 1997), YF476 and YM022 are high affinity canine and human CCK1 receptor antagonists.

There was significant complexity observed within the data from the radioligand binding studies although in each case it appeared to be ligand rather than species or receptor dependent. Thus, the saturation binding of [¹²⁵I]-BH-CCK-8S did not appear to plateau and also appeared biphasic in each assay. It is believed that this was a consequence of using an agonist radioligand, which labeled multiple agonist-induced states of the receptor. Indeed, one of the first studies to utilize this radioligand reported two affinity states in rat pancreatic acini (Sankaran et al., 1980, J. Biol. Chem., 255:1849-1853) with an estimated pK_{D} value for the high affinity site which was not significantly different to that obtained in this study (˜10.2 and ˜10.5, respectively). In addition to the biphasic nature of the saturation binding isotherm, it was also observed that the slope of the competition-inhibition curve for L-364,718 was significantly greater than unity in the assays of both the canine and human CCK1 receptor. Steep Hill slopes have been previously reported for L-364,718 in rat pancreatic tissue (n_{H}=2.01, pK₁=9; see, e.g., Silvente-Poirot et al., 1993, Eur. J. Biochem., 212:529-538). The slope for L-364,718 was not steep when measured at the canine CCK2 receptor, although due to the 10-fold lower affinity for this receptor the competition-inhibition curve was obtained over a higher concentration range. This data reflect the saturable depletion of L-364,718 at the lower values of the CCK1 receptor blocking concentrations. Notwithstanding this finding, it is apparent from this study that L-364,718 expresses the same overall high affinity for cloned canine and human CCK1 receptors.

Due to the fact that no specific binding of [¹²⁵I]-BH-CCK-8S was measured using the canine CCK1 receptor variant #2 in both a cell number titration assay and a saturation binding assay, the pharmacological characterization of the variants was restricted to variant #1. The saturation analysis of the specific binding of [¹²⁵I]-BH-CCK-8S to variant #1 indicated that the K_{D} value for the radioligand was not significantly different to that estimated at the wild-type canine CCK1 receptor. In addition, the competition-inhibition studies at variant #1 and the wild-type canine CCK1 receptor revealed no significant differences in the affinity of the ligands evaluated. Using RT-PCR it was demonstrated that the expression of the canine CCK1 receptor was approximately 5-fold less in the HEK cells expressing the variant #2 compared to the wild-type canine CCK1 receptor. Therefore, the failure to detect specific [¹²⁵I]-BH-CCK-8S binding in the variant #2 assay seems to be attributable to the low expression rather than the variant expressing significantly lower affinity for the radiolabel. It was not possible to correlate the expression levels with Bmax values obtained from the saturation binding experiments, as the agonist radiolabel appeared to be labeling multiple, agonist-dependent states. An antagonist radioligand would need to be employed to provide reliable estimates of Bmax.

While the invention has been described above in reference to preferred embodiments and illustrative examples, it will be appreciated that the invention is intended not to be limited by the foregoing detailed description, but to be defined by the claims as properly construed under principles of patent law.

What is claimed is:

1. An isolated biologically active canine cholecystokinin 1 receptor polypeptide that is a polypeptide having an amino acid sequence as set forth in SEQ ID NO.:14 or SEQ ID NO.:15 or a functional variant thereof.

2. An isolated biologically active canine cholecystokinin 1 receptor polypeptide according to claim 1 having an amino acid sequence as set forth in SEQ ID NO.:14 or SEQ ID NO.:15.

3. An isolated canine cholecystokinin 1 receptor polypeptide having an amino acid sequence as set forth in SEQ ID NO.:16.

4. An isolated polynucleotide encoding a canine cholecystokinin 1 receptor polypeptide that is a polynucleotide having a nucleotide sequence as set forth in SEQ ID NO.:11 and SEQ ID NO.:12 or a complement thereof that hybridizes under stringent conditions thereto.

5. An isolated polynucleotide encoding a canine cholecystokinin 1 receptor polypeptide according to claim 4 having a nucleotide sequence as set forth in SEQ ID NO.:11 or SEQ ID NO.:12.

6. An isolated polynucleotide encoding a canine cholecystokinin 1 receptor polypeptide that is a polynucleotide having a nucleotide sequence as set forth in SEQ ID NO.:13 or a complement thereof that hybridizes under stringent conditions thereto.

7. A vector comprising a polynucleotide as defined in claim 4 operably linked to a promoter element that produces canine cholecystokinin 1 receptor RNA or expresses the canine cholecystokinin 1 receptor polypeptide encoded by said polynucleotide in a transfected host cell.

8. A vector as defined in claim 7, wherein the polynucleotide has a sequence as set forth in SEQ ID NO.:11 or SEQ ID NO.:12.

9. A recombinant host cell that has been transfected with a vector as defined in claim 7.

10. A recombinant host cell that has been transfected with a vector as defined in claim 8.

11. A method of identifying a compound that modulates cholecystokinin 1 receptor activity, comprising the steps of:
(a) contacting:
(i) a test sample comprising a compound, with
(ii) an assay reagent comprising: a receptor material expressing or comprising a biologically active canine cholecystokinin 1 receptor polypeptide having an amino acid sequence as set forth in SEQ ID NO.:14 or SEQ ID NO.:15 or a functional variant thereof, and a cholecystokinin 1 receptor ligand;
(b) determining the biological activity of the receptor after performing step
(a); and
(c) comparing the biological activity determined in step (b) with a control measurement obtained by contacting a control sample not containing the compound with the assay reagent.

12. A method as defined in claim 11, wherein said cholecystokinin 1 receptor ligand is selected from the group consisting of sulfated CCK-8, desulfated CCK-8, desulfated ¹²⁵I-BH-CCK-8, sulfated ¹²⁵I-BH-CCK-8, L-364,718, L-365,260, YF476, YM022, and dexloxiglumide.

13. A method as defined in claim 11, wherein said cholecystokinin 1 receptor ligand is a high-affinity ligand.

14. A method as defined in claim 11, wherein the receptor material is derived from a biological sample obtained from a dog.

15. A method of identifying a compound that binds to a biologically active canine cholecystokinin 1 receptor or a functional variant thereof, comprising the steps of:
(a) contacting a receptor material comprising or expressing a biologically active canine polypeptide having an amino acid sequence as set forth in SEQ ID NO:14 or SEQ ID NO.:15 or a functional variant thereof and a test compound with a labeled cholecystokinin 1 receptor ligand;
(b) determining the amount of the labeled cholecystokinin 1 receptor ligand that complexes with the receptor material; and
(c) comparing the amount determined in step (b) with a control measurement obtained by contacting the receptor material with the labeled cholecystokinin 1 receptor ligand in the absence of the test compound.

16. A method as defined in claim 15, wherein said cholecystokinin 1 receptor ligand is selected from the group consisting of sulfated CCK-8, desulfated CCK-8, desulfated ¹²⁵I-BH-CCK-8, sulfated ¹²⁵I-BH-CCK-8, L-364,718, L-365,260, YF476, YM022, and dexloxiglumide.

17. A method as defined in claim 15, wherein said cholecystokinin 1 receptor ligand is a high-affinity ligand.

18. A method as defined in claim 15, wherein the receptor material is derived from a biological sample obtained from a dog.

19. A whole cell method to assay a compound for modulation of canine cholecystokinin 1 receptor activity, comprising:
(a) contacting a compound with a cell comprising or expressing a biologically active cholecystokinin 1 receptor polypeptide having an amino acid sequence as set forth in SEQ ID NO:14 or SEQ ID NO.:15 or a functional variant thereof; and
(b) determining any change in the cell in response to modified receptor function by the compound.

20. A method as defined in claim 19, wherein said determining any change comprises measuring: directly for a change in the activity, function or quantity of said receptor; for a downstream effect of the receptor function; or for a phenotypic change in the cell.
